(19) United States
(12) Patent Application Publication
(10) Pub. No.: US 2016/0370376 A1
(43) Pub. Date: Dec. 22, 2016

(54) COMPOUNDS AND METHODS FOR DETECTION AND ISOLATION OF BIOMOLECULES

(71) Applicant: ANDREI POLUKHTIN, SCOTTSDALE, AZ (US)

(72) Inventors: Andrei POLUKHTIN, Scottsdale, AZ (US); Patrick L WALSH, Chandler, AZ (US)

(73) Assignee: ANDREI POLUKHTIN, SCOTTSDALE, AZ (US)

(21) Appl. No.: 14/789,784

(22) Filed: Jul. 1, 2015

Related U.S. Application Data
(63) Continuation-in-part of application No. 14/742,403, filed on Jun. 17, 2015, now abandoned.

(51) Int. Cl. G01N 33/58; C07F 9/553; C07D 403/12; C07D 257/08; C07D 207/46; C07D 225/08

(52) U.S. Cl. CPC G01N 33/583; C07D 207/46; C07D 225/08; C07D 403/12; C07D 257/08; C07F 9/5535; G01N 2458/00

(57) ABSTRACT

A compound of general formula R₁-L₁-PCL-L₂-R₂ is disclosed wherein PCL is a photolabile group; R₁ is a reactive moiety capable of modifying biomolecules without activation; L₁ is a non-cleavable linker or absent; L₂ is a non-cleavable linker or absent; R₂ is a reactive moiety partner of a pair of orthogonally reactive moieties that can react with each other in the presence or absence of a catalyst without activation and both reactive moieties are sufficiently stable under commonly applied biomolecule labeling conditions. In another embodiment of the compound, R₁ is a biomolecule. A kit comprising at least one of these compounds and a method that uses these compounds are also disclosed.

### BACKGROUND OF THE INVENTION

1. Field of the Invention

This invention relates to agents and conjugates used in the detection and isolation of targets from heterologous mixtures. The agents comprise a bioorthogonal moiety bound to a photoreactive moiety. The conjugates comprise agents, which are coupled to substrates by one or more covalent bonds. These bonds can be easily and selectively cleaved or photocleaved with the application of electromagnetic radiation. Substrates, which may be coupled to the agents, include amino acids, peptides, proteins, nucleotides, nucleic acid primers and lipids. The invention also relates to rapid and efficient methods for the detection and isolation of targets, such as cells, nucleic acids and proteins, and to kits, which contain these components.

2. Description of the Background

There currently exists a wide variety of techniques and approaches for the detection and isolation of a specific target from a complex mixture. Methods such as chromatography or gel electrophoresis are commonly used in the separation of biomolecules from a complex mixture. These techniques take advantages of a distinct chemical or physical attribute of the target molecule such as charge, hydrophobicity, or molecular weight, among others. Some of the more useful detection and isolation procedures take advantage of physical properties of the species of interest, the substrate, or of molecules, which can be easily attached to substrates. While there are numerous labeling strategies, based on different detection principles, the most useful of these allow the target molecule to be specifically identifiable from all others present in the mixture.

One example of a process to render a substance specifically detectable is to use binding molecules, which have a particular affinity for selected other molecules, such as that which occurs through the binding of an antigen to an antigen-specific antibody. Usually, one of the molecules in this pair (either the antigen or antibody) is immobilized on a solid support, such as used in chromatographic packing material or a magnetic bead, and used in the isolation of the target molecule. Some of the more useful coupling agents are biotin and avidin or the related protein, streptavidin. These agents have been used in many separation techniques to facilitate isolation of one component or multiple components from complex mixtures.

Biotin, a water-soluble vitamin, is used extensively in biochemistry and molecular biology for a variety of purposes including macromolecular detection, purification and isolation, and in cytochemical staining. Biotin also has important applications in medicine in the areas of clinical diagnostic assays, tumor imaging and drug delivery, and is used extensively in the field of affinity cytochemistry for the selective labeling of cells, subcellular structures and proteins.

Biotin's usability stems from its ability to bind strongly and selectively to the tetrameric protein avidin, found in egg white and the tissues of birds, reptiles and amphibians, or to its chemical cousin, streptavidin, isolated from the bacterium Streptomyces Avidinii. Typically, biotin or a derivative of biotin is first bound directly to a target molecule, such as a protein or oligonucleotide, or to a probe using specific chemical linkage. The interaction of the linked biotin with either streptavidin or avidin conjugated to an affinity medium such as magnetic or agarose beads is then used in the isolation of the target molecule. Alternatively, the interaction of the covalently linked biotin with avidin or streptavidin conjugated to an enzyme such as horseradish peroxidase (HRP), which catalyzes a chromogenic reaction, is used for detection of the target molecule.

While the utility of biotin continues to grow, there still exist major drawbacks in the use of biotin-streptavidin technology for many applications. This problem stems from the high affinity between biotin and streptavidin, precisely the molecular characteristic which makes it most useful. Once a target molecule or cell is isolated through the streptavidin-biotin interaction, release of the target molecule requires disruption of this interaction. Dissociation of biotin from streptavidin requires very harsh conditions, such as 6-8 molar (M) guanidinium-HCl, pH 1.5. However, such conditions also denature, and thereby inactivate most proteins and destroy cells. For example, a biotin derivative containing an N-hydroxysuccinimide ester group is commonly used to link biotin through an amide bond to proteins and nucleic acids. Selective cleavage of this linkage also disrupts similar native chemical bonds in associated molecules. Biotin is also often used in the isolation of specific cells from a heterogeneous mixture of cells by binding a biotinylated antibody directed against a characteristic cell surface antigen. The interaction of the biotinylated antibody with streptavidin-coated magnetic beads or sepharose particles can then be used effectively to isolate target cells. Disruption of the antibody-antigen interaction normally requires exposure of cells to conditions such as low pH or mechanical agitation, which are adverse to the cell's survival. In general, recovery of the target in a completely unmodified form is not possible.

Once biotinylated DNA is bound to streptavidin, it can only be released with extreme difficulty. Many diverse methods to remove the streptavidin molecule have been suggested including digestion by proteinase K (M. Wilchek and E. A. Bayer, Anal. Biochem. 171:1, 1988). However, proteinase K also digests nearby proteins and does a fairly poor job of completely digesting the streptavidin. Significant amounts of the streptavidin molecules remain attached, and further removal of streptavidin does not release the biotin. Furthermore, biotinylated DNA interferes with subsequent use of the DNA in a variety of methods, including transformation of cells and hybridization based assays used for detection of genetic diseases.

The essentially irreversible binding of biotin and streptavidin is also a serious limitation for the performance of multiple or sequential assays to detect a specific type of biomolecule, macromolecular complex, virus or cell present in a single sample. Normally, only a single assay can be performed because the enzyme detection system is streptavidin-based and streptavidin remains firmly bound to the biotinylated target or target probe. While different chromogenic systems for detection are available, they are only of limited applicability in situations where large numbers of probes are needed.

An additional problem in the use of biotin-avidin technology is the presence of endogenous biotin, either free or complexed to other molecules, inside the sample to be purified or assayed. In this case, the endogenous biotin, which is present in many biological samples, can result in the isolation or detection of non-target molecules. This can be a particularly severe problem in cases where a high signal-to-noise ratio is needed for accurate and sensitive detection.

To remove biotin from an attached molecule, several chemically cleavable biotin derivatives have been produced. IMMUNOPURE NHS-SS-biotin (Pierce Chemical; Rockford, Ill.) consists of a biotin molecule linked through a disulfide bond and an N-hydroxysuccinimide ester group that reacts selectively with primary amines. Using this group, NHS-SS-biotin can be linked to a protein and then the biotin portion removed by cleaving the disulfide bond with thiols. However, this approach is also of limited use since thiols normally disrupt native disulfide bonds in proteins. Furthermore, the cleavage still leaves the target cell or molecule modified since the spacer arm portion of the complex, including one of the thiols, is not removed and the cleaving buffer must be eliminated from the sample. Once cleaved, the thiol which remains can cause homo-dimerization or reaction with thiols present in the target molecule. Functional activity of these substances containing sulfhydryl groups is severely compromised. Typically, activity of such proteins is decreased or eliminated and such nucleic acids will no longer hybridize, rendering them useless for cloning. This method is also slow and requires the preparation of complex solutions.

An additional limitation of biotin-avidin technology is the difficulty of developing automated systems for the isolation and/or detection of targets due to the problems of releasing the target from the biotin-avidin binding complex. This requires addition of specific chemical reagents and careful monitoring of the reactions.

Biotin-avidin technology has been combined with PCR techniques for the detection and isolation of nucleic acids and specific sequences. However, there still remains a fundamental problem, which relates to the difficulty of removing the incorporated biotin. This is normally not possible using conventional biotins without irreversibly altering the structure of the DNA. As discussed, biotinylation can interfere with subsequent application of biotinylated probes as well as alter the properties of the PCR product.

PCR products that contain biotinylated nucleotides or primers, which are required for isolation, cannot be used in conjunction with biotinylated hybridization probes. The presence of biotin on the PCR product causes false signals from the avidin based enzyme-linked detection system. Biotin incorporation into DNA also interferes with strand hybridization possibly due to the spacer arms linking the nucleotides to the biotin molecules. Further, PCR products that are biotinylated are not suitable material for cloning. PCR products, which contain biotinylated nucleotides, are difficult to analyze. Incorporation of biotinylated nucleotides into DNA causes a retardation of mobility during agarose gel electrophoresis. This mobility shift renders characterization of PCR products difficult. As proper DNA-DNA hybridization is the basis for sensitive and accurate characterization and sensitive assays, biotin-avidin binding systems are seriously disadvantaged.

Unlike conventional biotins, photocleavable biotins enable one to release or elute the bound substrate from the immobilized avidin, streptavidin or their derivatives in a completely unmodified form. This is extremely useful and an important improvement over non-photocleavable biotins for a number of reasons. Biotinylation of the target material can impede its subsequent use or characterization. Biotinylation of a protein can alter its activity, electrophoretic mobility, ability to bind a substrate, antigenicity, ability to reconstitute into a native form and ability to form multisubunit complexes. In contrast, by using photocleavable biotin, once the biotin is photocleaved from the protein or protein/binding complex, all the native properties and function will be restored to the native form for further use and characterization.

Among the many methods for the detection and isolation of biomolecules, photocleavable reagents have strong utility due to their selective cleavage ability. There are a number of photocleavable groups which can be attached to different reactive moieties. The more common bioreactive groups can act on amines, alcohols, carboxylic, phosphates, among others. The detectable moiety can either be directly coupled to the photocleavable reagent or can be subsequently detected. Directly detectable photocleavable reagents include those containing a fluorescent molecule such as coumarin or dansyl; indirectly detectable examples include those containing terminal amines, thiols and, the most usuable—biotin. Examples include photocleavable biotin NHS-ester, which selectively reacts with aliphatic amines to form an amide bond, and photocleavable biotin phosphoramidite, which can be incorporated into synthetic nucleotides, leaving a photocleavable biotin on the 5′ end of the oligonucleotide.

Although photocleavable biotin technology is an invaluable tool, it is not without drawbacks. The use of biotin, and therefore photocleavable biotin, as a source to isolate target molecules must be paired with streptavidin coupled to a solid support. Streptavidin-coated solid supports suffer from low binding capacity versus solid phases functionalized with small reactive groups. For example, commercially available magnetic beads containing carboxy or amine functional groups usually contain 600 to 650 nmole of reactive groups per milligram of magnetic beads. The highest capacity of streptavidin beads commercially available is about 3.5 nmole of streptavidin per mg—a 180 fold decrease in binding capacity. Streptavidin agarose resins suffer from similar issues; the highest capacity commercially available streptavidin agarose contains around 300 nmole/ml of biotin binding sites, whereas the same amine-functionalized resin has 25 μmol/ml of available amines (around 80 fold decrease in binding capacity). An additional drawback associated with the use of streptavidin solid supports is the possible leaching of streptavidin. The unwanted release of streptavidin from the solid support can occur under certain conditions and leads to strong protein background signal, loss of binding capacity and the loss of target molecules.

One solution to the issues mentioned above is to provide agents, which can detect and isolate targets with specificity, have a high binding capacity and allow selective release. As discussed above, the use of biotin for target isolation is associated with a number of drawbacks, such as the low binding capacity of streptavidin solid supports. This could be circumvented by using smaller moieties reactive towards the detectable group, such as thiols and amines with maleimides and NHS esters, respectively. Despite the possibility for increased binding capacity, these detectable moieties come with a number of disadvantages. The use of a thiol as a detectable entity is unwanted due to the nucleophilic nature of the sulfur group, which can cross-react with nascent thiols and disulfides present in many biological samples. Furthermore, the use of maleimide reactive groups for detection of these thiols is pH dependent—conjugation can occur with primary amines at pH greater than 6.5. Maleimides also lack specificity as the reaction can proceed with any reduced thiol present in the mixture. The use of primary amines—NHS coupling chemistry which is common and relatively efficient, still has some drawbacks as well. The use of primary amines has reduced utility due to the fact that most biological samples contain numerous primary amines, reducing the specificity of the isolation. Additionally, the competing hydrolysis of NHS esters makes it necessary to use large excesses of NHS ester reagent which must be removed from the reaction and thus is counterproductive in obtaining pure target samples.

The solution to the issues discussed above is to utilize highly selective bioorthogonal chemistry. The term bioorthogonal chemistry refers to any chemical reaction that can occur in the presence of rich functionalities found in biological media without interfering with native biochemical processes. In this strategy, a small linker containing bioorthogonal functionality is introduced onto a biomolecule through a photolabile linker. The capturing resin, which is equipped with a complementary functional group reactive toward the linker, is then reacted with the protein-linker conjugate to yield the desired conjugate.

Agents of the invention consist of a bioorthogonal moiety and a photolabile group which can be covalently attached to a wide variety of substrates. Capture and cleavage conditions do not need to be developed for various substrates. Since the reactions of bioorthogonal groups, such as azide-alkyne cycloaddition, are well known and robust, the attachment of substrates is straightforward and should be identical for any and all substrates. Following attachment, electromagnetic radiation need only be applied to release the photocleavable moiety from the substrate.

### SUMMARY OF THE INVENTION

In a first embodiment, the present invention is directed to a compound of general formula:

R₁-L₁-PCL-L₂-R₂

wherein PCL is a photolabile group; R₁ is a reactive moiety capable of modifying biomolecules without activation; L₁ is a non-cleavable linker or absent; L₂ is a non-cleavable linker or absent; R₂ is a reactive moiety partner of a pair of orthogonally reactive moieties that can react with each other in the presence or absence of a catalyst without activation and both reactive moieties are sufficiently stable under commonly applied biomolecule labeling conditions. The photolabile group may be selected from the group consisting of:

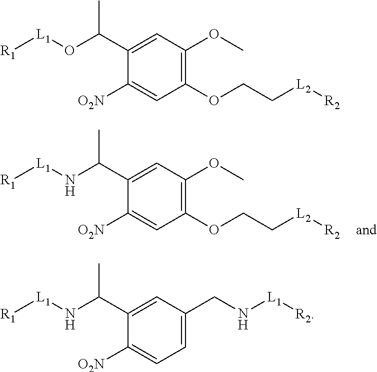

In the general formula, R₁ may be an amine reactive activated ester or activated carbonate capable of modifying primary amine-containing biomolecules, selected from the group consisting of N-hydroxysuccinimidyl, sulfo-N-hydroxysuccinimidyl, 4-sulfo-2,3,5,6,-tetrafluorophenyl, 2,3,5,6-tetrafluorophenyl, —O-benzotriazole, benzotriazole carbonate, p-nitrophenyl carbonate, benzotrizole carbonate, 2,3,5-trichlorophenyl carbonate, and succinimidyl carbonate. In the alternative, R₁ may be a sulfhydryl reactive group capable of modifying sulfhydryl-containing biomolecules, selected from the group consisting of maleimdie, iodoacetyl, pyridildisulphide, vinylsulfone, and α,β-unsaturated carbonyl. As another option, R₁ may be a phosphoramidite group.

In the general formula, R₂ may be selected from the group consisting of orthogonal reactive pairs that can undergo Staudinger ligation, copper-catalyzed Huisgen 1,3-dipolar cycloaddition, strain-promoted Huisgen 1,3dipolar cycloaddition, Inverse Electron Demand Diels-Alder cycloaddition, and hydrazone or oxime bond forming reactions.

In a second embodiment, the present invention is directed to a kit comprising at least one compound described above in the first embodiment of the present invention.

In a third embodiment, the present invention is directed to a compound of general formula:

R₁-L₁PCL-L₂-R₂

wherein PCL is a photolabile group; R₁ is a biomolecule; L₁ is a non-cleavable linker or absent; L₂ is a non-cleavable linker or absent; R₂ is a reactive moiety partner of a pair of orthogonally reactive moieties that can react with each other in the presence or absence of a catalyst without activation and both reactive moieties are sufficiently stable under commonly applied biomolecule labeling conditions. The photolabile group may be selected from the group consisting of:

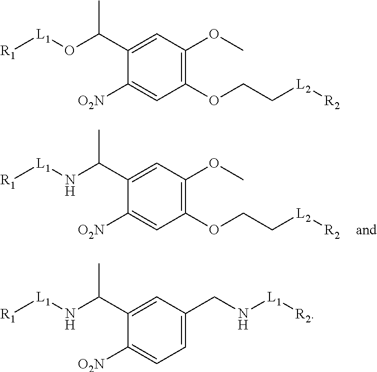

The biomolecule may be selected from the group consisting of a protein, peptide, amino acids, lipids, cells, virus particle, fatty acids, polysaccharides, and synthetic or natural, single or double strand DNA or RNA. In the general formula, R₂ may be selected from the group consisting of orthogonal reactive pairs that can undergo Staudinger ligation, copper-catalyzed Huisgen 1,3-dipolar cycloaddition, strain-promoted Huisgen 1,3dipolar cycloaddition, Inverse Electron Demand Diels-Alder cycloaddition, and hydrazone or oxime bond forming reactions.

In a fourth embodiment, the present invention is directed to a kit comprising at least one compound described above in the third embodiment of the present invention.

In a fifth embodiment, the present invention is directed to a method, comprising:
modifying a biomolecule with the compound of the first embodiment of the present invention to produce a conjugate, wherein the conjugate is a bioorthogonal moiety covalently linked to a biomolecule through a photolabile linker;
reacting the conjugate with a solid support or another molecule functionalized with a complementary bioorthgonal moiety; and
treating the conjugate under conditions such that the conjugate is photocleaved and the biomolecule is released.

The biomolecule may be a synthetic oligonucleotide, and the modifying step may be done during a solid phase synthesis.

In a sixth embodiment, the present invention is directed to a method, comprising:
modifying a biomolecule with the compound of the first embodiment of the present invention to produce a conjugate, wherein the conjugate is a bioorthogonal moiety covalently linked to a biomolecule through a photolabile linker; and
reacting the conjugate with a solid support or another molecule functionalized with a complementary bioorthgonal moiety.

The biomolecule may be a synthetic oligonucleotide, and the modifying step may be done during a solid phase synthesis.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to one of ordinary skill in the art from this detailed description.

### BRIEF DESCRIPTION OF THE FIGURES

The present invention will become more fully understood from the detailed description given below and the accompanying drawings that are given by way of illustration only and are thus not limitative of the present invention.

FIG. 1 illustrates the mechanism of photocleavage in 2-nitrobenzyl-1-based systems.

FIG. 2 is a schematic representation of the isolation of biomolecules using photocleavable biotin. An amine containing biomolecule is labeled with photocleavable biotin-NHS ester followed by capturing of said labeled protein onto streptavidin resin. The captured biomolecule is released by irradiating the resin with 350 nm light.

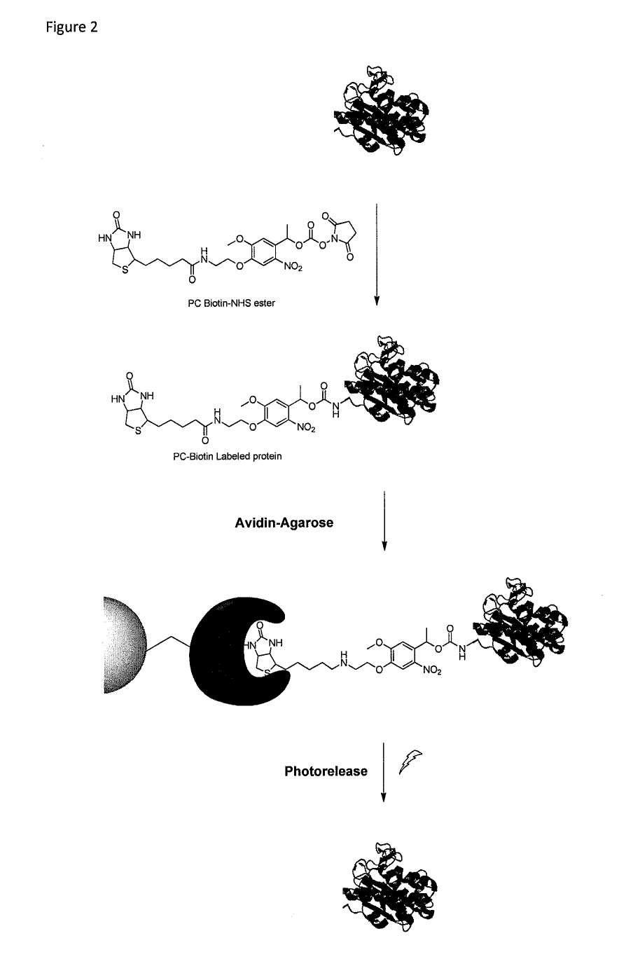

FIG. 3 is a schematic representation of the isolation of biomolecules using photocleavable tetrazine and agarose-TCO. An amine containing biomolecule is labeled with photocleavable tetrazine-NHS ester followed by capturing of said labeled protein onto trans-cyclooctene modified resin. The captured biomolecule is released by irradiating the resin with 350 nm light.

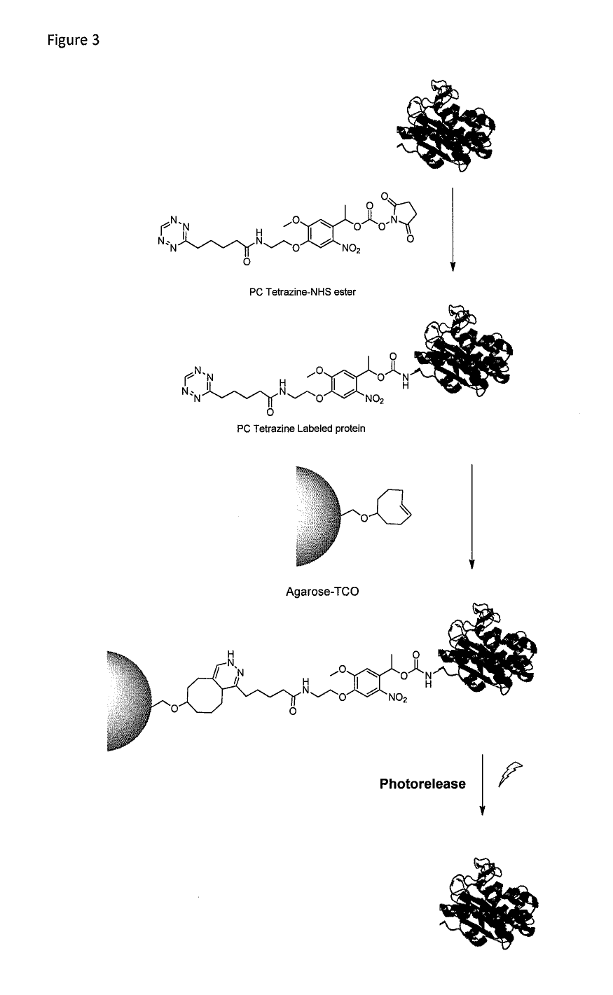

FIG. 4 is a schematic representation of the isolation of target protein and antibody using photocleavable tetrazine and agarose-TCO. An amine containing antigen is labeled with photocleavable tetrazine-NHS ester followed by the incubation of the said labeled antigen with an antibody and capturing of antigen-antibody complex onto trans-cyclooctene modified resin. The captured complex is released by irradiating the resin with 350 nm light.

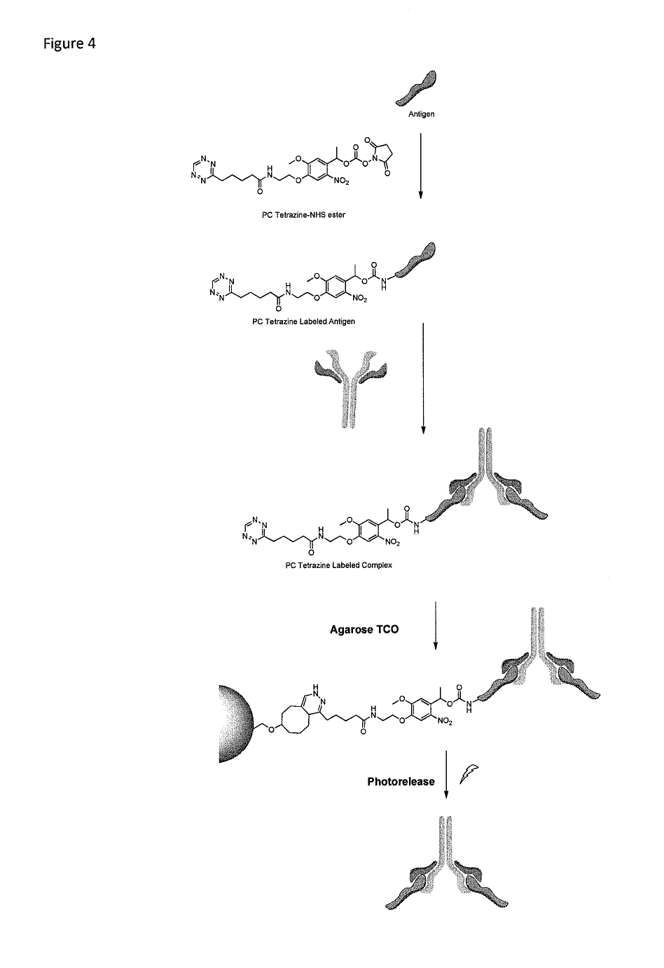

FIG. 5 is a schematic representation of the isolation and hybridization of nucleotides. A single strand, amine-containing oligonucleotide is labeled with photocleavable tetrazine-NHS ester followed by hybridization with complementary oligonucleotide and capturing of said complex onto trans-cyclooctene modified resin. The captured complex is released by irradiating the resin with 350 nm light

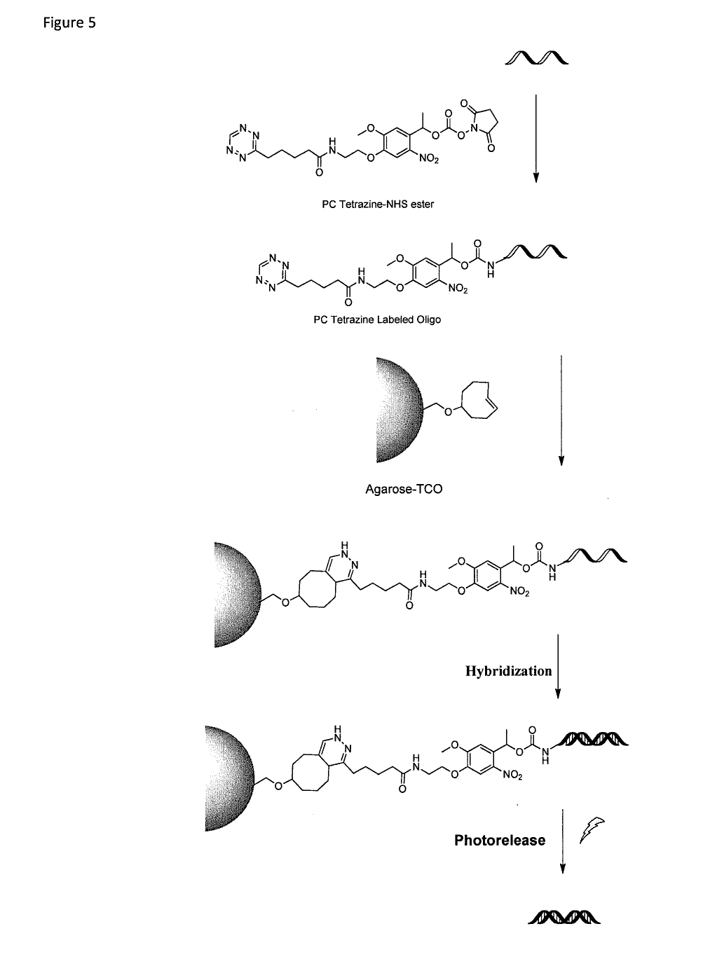

FIG. 6 is a schematic representation of the purification of a full-length target sequence with a DBCO-azide ligation pair. A short synthetic oligonucleotide is labeled with photocleavable DBCO during solid phase oligonucleotide synthesis. The photocleavable DBCO-modified oligonucleotide is captured onto azide-modified resin. The captured oligonucleotide is released by irradiating the resin with 350 nm light

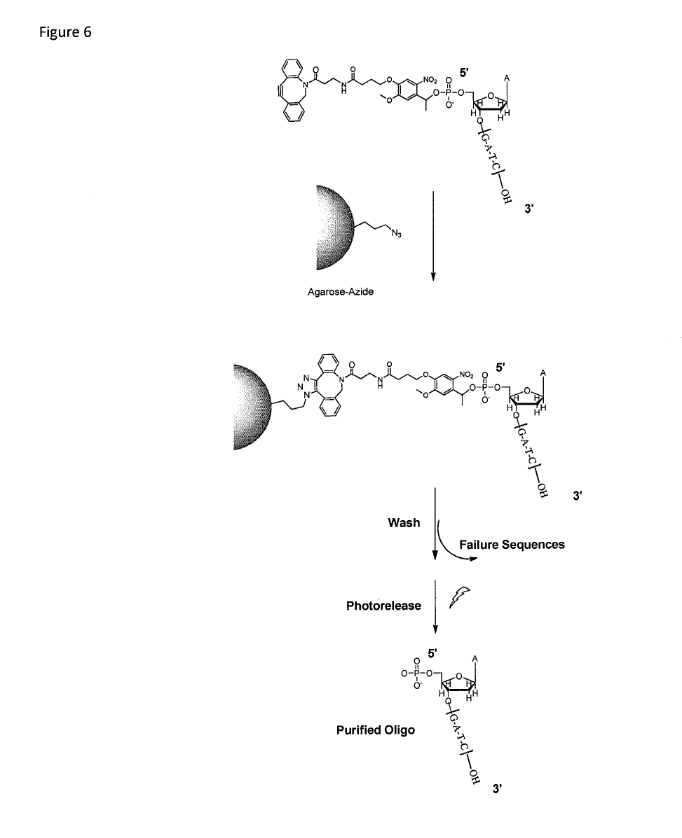

FIG. 7 is SDS-PAGE analysis of protein catch and release. Beta-lactoglobulin was labeled with photocleavable tetrazine-NHS ester and spiked into a mixture of several proteins. The mixture of proteins were treated with trans-cyclooctene modified resin followed by two washes to remove non-specifically bound proteins. The target protein is released by irradiating the resin with 350 nm light. The same procedure was performed using photocleavable DBCO-NHS ester.

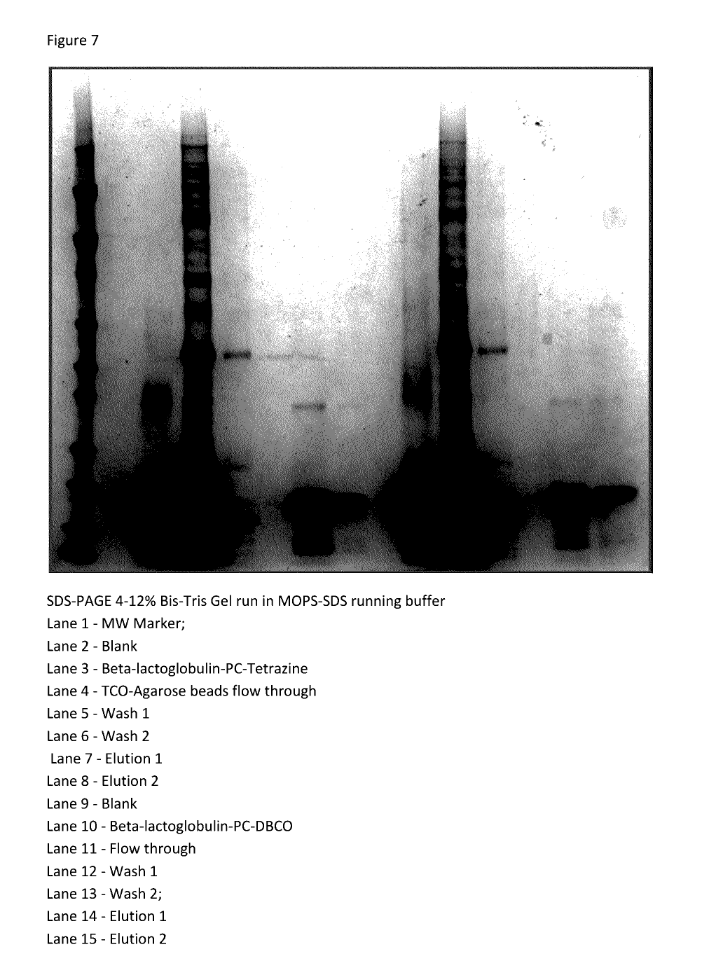

### DETAILED DESCRIPTION OF THE INVENTION

The present invention will now be described with reference to the accompanying drawings.

The present invention is directed to compositions and methods used in the detection and isolation of biomolecules, cells and any identifiable substance from a mixture. Compounds are comprised of a bioorthogonal reactive group covalently bound to a reactive moiety through a photocleavable moiety. Conjugates comprise agents which are coupled to substrates by one or more covalent bonds which, by the presence of the photocleavable moiety, are selectively cleavable with electromagnetic radiation. The invention is also directed to methods for the isolation and detection of targets using these compounds and conjugates and to kits which utilize these methods for the detection and isolation of nearly any substance from a heterologous mixture.

Unless defined otherwise, all terms used herein have the same meaning as is commonly understood by one of ordinary skill in the art to which this invention belongs. All patents, patent applications and publications referred to throughout the disclosure herein are incorporated by reference in their entirety. In the event that there is a plurality of definitions for a term herein, those in this section prevail.

The term “biomolecule” as used herein refers to a compound of biological origin or of biological activity. Biomolecules include for example a nucleic acid, a nucleotide, a protein, an amino acid, a carbohydrate monomer and a polysaccharide. If the biomolecule is a nucleic acid, it may be DNA, cDNA, RNA, or PNA and may comprise natural or unnatural bases or internucleotide linkages, such as phosphodiesters, phosphorothioates, phosphoramidites or peptide nucleic acids.

The term “reactive moiety” or “reactive group” herein refers to a moiety that can be coupled with another moiety without prior activation or transformation. Some commercially sold molecules referred to herein as linking moieties include those that react with free amines on the target molecule, such as N-hydroxysuccinimidyl, p-nitrophenyl, pentafluorophenyl and N-hydroxybenzotriazolyl ester, and those that react with free sulfhydryls present on the target molecule such as maleimido, alpha-haloacetamido and pyridyldisulfides.

The term “linker” is a covalent linkage having 1-48 nonhydrogen atoms selected from the group consisting of C, N, O, P, and S and composed of any combination of single, double, triple or aromatic carbon-carbon bonds, carbon-nitrogen bonds, nitrogen-nitrogen bonds, carbon-oxygen bonds, carbon-sulfur bonds, phosphorus-oxygen bonds, and phosphorus-nitrogen bonds.

The term “bioorthogonal chemistry” refers to any chemical reaction that can occur in the presence of rich functionalities of biological media without interfering with native biochemical processes.

The term “protecting group” refers to a group that temporarily protects or blocks, i.e., intended to prevent from reacting, a functional group, e.g., an amino group, a hydroxyl group, or a carboxyl group, during the transformation of a first molecule to a second molecule.

The term “ligand/receptor couple” as used herein refers to a pair of molecules having a substantially high affinity for binding specifically to one another. One example of such a binding pair would be a receptor on a cell and the ligand that binds that receptor. Another example would be biotin and avidin, which are two molecules that have a strong affinity for binding each other and having an association constant of around 10¹⁵. Other pairs include Peptide S and ribonuclease A, digoxigenin and its receptor and oligonucleotides with complimentary sequences.

Various methods exist which may be employed to bind the extended linking group to a macromolecule or fragment. For example, to facilitate this binding, the extended linking group may be attached to biomolecule-reactive groups, such as active ester groups, amino groups, sulfhydryl groups, carbohydrate groups, azido groups or carboxy groups. A variety of methodologies exist for reacting biomolecule-reactive groups with macromolecules or macromolecule fragments. Examples of such methodologies are photo-crosslinking and glutaraldehyde crosslinking Still other methods for affecting such coupling will occur to those skilled in the art. See, for examples of such methods: Hermanson, G. T., Bioconjugate Techniques, Elsevier Science, London, 2008.

One embodiment of the invention relates to compounds of general Formula I comprising a reactive bioorthogonal moiety bound to a reactive moiety through a photocleavable moiety where the reactive moiety is capable of covalently bonding a biomolecule to form a conjugate. This conjugate can subsequently be selectively cleaved to release the substrate or, alternatively, to release any group or molecule attached to the photocleavable moiety. Cleavage, as referred to herein, is by photocleavage or a cleavage event triggered by the application of radiation to the conjugate. The radiation applied may comprise one or more wavelengths from the electromagnetic spectrum but is generally required in the UV-range (10 to 380 nm). Various and multiple sources of irradiation may be applied in a simultaneous or alternating fashion. The irradiation time may be for a time period of seconds, minutes or hours, either constant or in intervals.

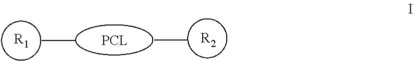

wherein
R₁ is a reactive bioorthogonal group;
PCL is a photocleavable moiety; and
R₂ is a reactive group that is capable of modifying biomolecules.

In preferred embodiments, the reactive groups that are capable of modifying biomolecules are activated esters. In more preferred embodiments, activated esters are NHS ester and PFP esters. To one of ordinary skill in the art, it will be apparent that there are multiple variations of reactive groups useful for modifying a biomolecule. Exemplary reactive groups are given in Table 1.

TABLE 1

| Reactive Group | Target Functional Group |
|---|---|
| Carbodiimide | Amine/Carboxyl |
| Carbonyl | Hydrazine |
| Diazirine | Nonselective |
| Hydrazide | Carbohydrate (oxidized) |
| Hydroxymethyl Phosphine | Amine |
| Imidoester | Amine |
| NHS-ester | Amine |
| PFP-ester | Amine |
| Psoralen | Amine |
| Pyridyl Disulfide | Sulfhydryl |
| Vinyl Sulfone | Sulfhydryl, amine, hydroxyl |
| Terminal Alkyne | Azide |
| Azide | Terminal alkyne, cyclooctyne |
| Trans-Cyclooctene | Tetrazine |

Another embodiment of the invention relates to compounds of the general formula:

R₁-L₁-PCL-L₂-R₂

wherein PCL is a photolabile group;
R₁ is (i) a reactive moiety capable of modifying biomolecules without activation or (ii) a biomolecule;
L₁ is a non-cleavable linker or absent;
L₂ is a non-cleavable linker or absent;
R₂ is a reactive moiety partner of a pair of orthogonally reactive moieties that can react with each other in the presence or absence of a catalyst without activation and both reactive moieties are sufficiently stable under commonly applied biomolecule labeling conditions.

Examples of the photolabile group for PCL may include o-alkylated nitrophenyl compounds, o-alkylated aryl ketones, a benzoin group, a p-hydroxyphenacyl group, a coumarinyl group, or α-substituted methylphenols (see for review A. P. Pelliccioli and J. Wirz, Photochem. Photobiol. Sci. 1:441, 2002). The photolabile group is preferably o-alkylated nitrophenyl group.

Examples of the reactive moiety capable of modifying biomolecules without activation for the R₁ group may include an amine reactive activated ester or activated carbonate capable of modifying primary amine-containing biomolecules or a sulfhydryl reactive group capable of modifying sulfhydryl-containing biomolecules or a phosphoramidite group for modifying oligonucleotides during solid phase synthesis. R₁ may be —O—, —OH, —SH, —NH, —NH₂, —F, —Cl, —Br, —I, O-Su (i.e., N-hydroxysuccinimidyl or sulfo-N-hydroxysuccinimidyl), —O-STP (i.e., 4-sulfo-2,3,5,6,-tetrafluorophenyl), —O-TFP (i.e., 2,3,5,6-pentafluorophenyl), —O-benzotriazole, benzotriazole, benzotriazole carbonate, p-nitrophenyl carbonate, benzotrizole carbonate, 2,3,5-trichlorophenyl carbonate, succinimidyl carbonate, —COCl, —SO₂Cl, —CO—CH₂—I, —COO—, —COOH, —CO—NH—NH₂, —O-phosphoramidite, —CHO, -maleimide, or pyridylsulfide. This reactive moiety is preferably N-hydroxysuccinimidyl, sulfo-N-hydroxysuccinimidyl, 4-sulfo-2,3,5,6,-tetrafluorophenyl, 2,3,5,6-tetrafluorophenyl, —O-benzotriazole, benzotriazole carbonate, p-nitrophenyl carbonate, benzotrizole carbonate, 2,3,5-trichlorophenyl carbonate, succinimidyl carbonate, maleimide, iodoacetyl, pyridildisulphide, vinylsulfone, or α,β-unsaturated carbonyl. This reactive moiety is more preferably N-hydroxysuccinimidyl, succinimidyl carbonate, or maleimide.

Examples of the biomolecule for the R₁ group may include a protein, peptide, amino acids, lipids, cells, virus particle, fatty acids, polysaccharides, or synthetic or natural, single or double strand DNA or RNA. The biomolecular is preferably protein or synthetic single strand DNA. The need for a coupling agent or a catalyst for the labeling reaction depends on the nature of the reaction and is generally well known to one skilled in the art.

The term “non-cleavable” means a covalent linkage that is substantially stable under conditions generally applied for storage, modification and usage of biomolecules. Examples of a non-cleavable linker for the L₁ and L₂ group may include a covalent linkage having 1-48 nonhydrogen atoms selected from the group consisting of C, N, O, P, and S and composed of any combination of single, double, triple, or aromatic carbon-carbon bonds, carbon-nitrogen bonds, nitrogen-nitrogen bonds, carbon-oxygen bonds, carbon-sulfur bonds, phosphorus-oxygen bonds, and phosphorus-nitrogen bonds. The non-cleavable linker is preferably (i) a divalent linear —(CH₂)ₓ— group or a —(CH₂CH₂O)ₓ— group wherein x is 1 to 25, (ii) a branched or cyclic alkane group, which is optionally substituted by at least one atom selected from the group consisting of oxygen, substituted nitrogen, and sulfur, or (iii) absent. The non-cleavable linker is more preferably an alkyl having 1-6 carbon atoms or a discrete or non-discrete polyethylene glycol linker. The non-cleavable linker for the L₁ group may be the same or different from the non-cleavable linker for the L₂ group.

Examples of the reactive moiety partner of a pair of orthogonally reactive moieties that can react with each other in the presence or absence of a catalyst without activation for the R₂ group may include orthogonal reactive pairs that can undergo Staudinger ligation, copper-catalyzed Huisgen 1,3-dipolar cycloaddition, strain-promoted Huisgen 1,3 dipolar cycloaddition, Inverse Electron Demand Diels-Alder cycloaddition, or hydrazone or oxime bond forming reactions. This reactive moiety partner is preferably cyclooctyne-azide and tetrazine-trans-cyclooctene (TCO) bioorthogonal reactive pairs.

Preferred examples of the compound include:

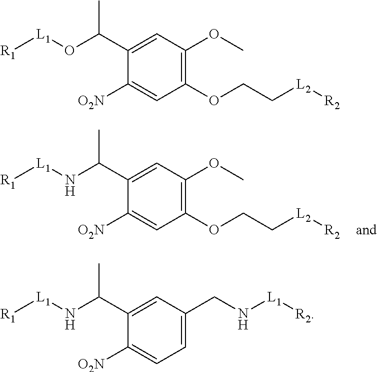

In still another aspect, the present invention relates to bio-orthogonal chemistry. The term bioorthogonal chemistry refers to any chemical reaction that can occur in the presence of rich functionalities of living systems/biological media without interfering with native biochemical processes. In this strategy, one component of the conjugate is modified with a bioorthogonal functional group, while in a separate reaction, the other component is activated with a complementary functional group of the bioorthogonal ligation pair. The two bioorthogonally-activated components are then mixed together and spontaneously react to form the specific conjugate. In certain embodiments, the bio-orthogonal reaction is a Cu-catalyzed version of Huisgen 1,3-dipolar cycloaddition between an azide and terminal alkyne. In other embodiments, the reaction is carried out in the absence of such a catalyst. Exemplary 1,3-dipole-functional compounds include, but are not limited to, azides, nitrile oxides, nitrones, and diazo compounds.

In preferred embodiments the reactive bioorthogonal pair is chosen to undergo Staudinger ligation, Cu-catalyzed 1,3-cycloaddition (click reaction), Cu-free click reaction, Inverse Demand Diels-Alder cycloaddition, or hydrazone or oxime bond forming reactions.

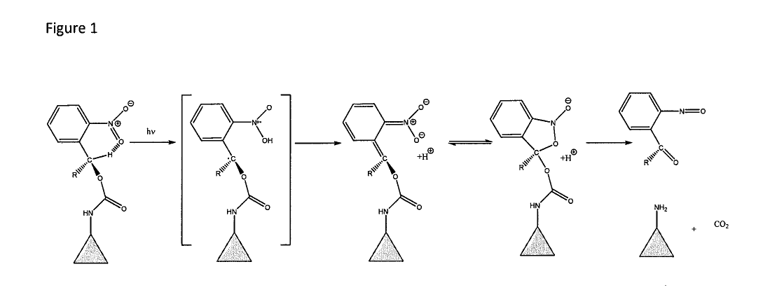

One class of photoreactive moieties are 2-nitrobenzyl derivatives. In their ground state, 2-nitrobenzyl-based compounds and conjugates have an intramolecular hydrogen bond between benzylic hydrogen and the ortho nitro group (—CH . . . O₂N) (B. Brzezinski et al., J. Chem. Soc. Perkin. Trans. 2:2257-61, 1992). Upon irradiation with wavelengths of greater than 300 nm, these chemical compounds transition to an excited state. Proton transfer reaction from benzylic carbon to the oxygen in nitro group takes place which is followed by electron rearrangement (FIG. 1). This reaction results in the formation of a transient species called an aci-nitro ion which is in a rapid equilibrium with a cyclic form. In the cyclic intermediate, electron rearrangement and oxygen transfer from nitrogen to benzylic carbon take place, resulting in the formation of 2-nitroso derivatives and release of a substrate, which is a good leaving group (J. A. McCray et al., Annu. Rev. Biophys. Chem. 18:239-70, 1989).

A variety of ortho-benzyl compounds as photolabile groups have been used in synthesis of both peptides (see Fodor et al. Science 251:767-773, 1994) and oligonucleotides (see Pease et al. Proc. Natl. Acad. Sci. USA 91:5022-5026). See PCT patent publication Nos. WO 90/15070, WO 92/10092, and WO 94/10128; see also U.S. patent application Ser. No. 07/971,181, filed 2 Nov. 1992, and Ser. No. 08/310,510, filed Sep. 22, 1994; Holmes et al. (1994) in Peptides: Chemistry, Structure and Biology (Proceedings of the 13th American Peptide Symposium); Hodges et al. Eds.; ESCOM: Leiden; pp. 110-12, each of these references is incorporated herein by reference for all purposes. Examples of these compounds included the 6-nitroveratryl derived protecting groups, which incorporate two additional alkoxy groups into the benzene ring. Introduction of an α-methyl onto the benzylic carbon facilitated the photolytic cleavage with greater than 350 nm UV light and resulted in the formation of a nitroso-ketone.

One of ordinary skill in the art will recognize that there are multiple variations of photolabile groups that could be used for the purpose of this invention.

Preferred embodiments are those in which the photocleavable group has formulas:

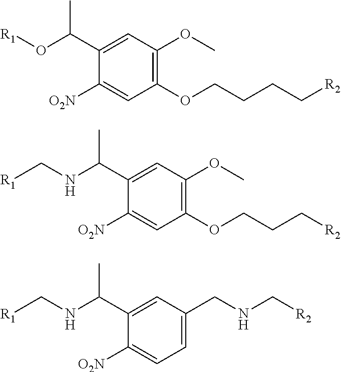

wherein
R₁ is a reactive bioorthogonal group; and
R₂ is a reactive group that is capable of modifying biomolecules.

Generally, photocleavage is achieved by placing the source of electromagnetic radiation (such as a lamp) at a specified distance from the sample to be irradiated. This distance can be calculated from the energy output of the device or empirically determined. The conjugate to be irradiated can be in solution or covalently attached to a solid support.

In most applications, the radiation applied is UV, visible or IR radiation of the wavelength between about 200 nm to about 1,000 nm, more preferably between about 260 nm to about 600 nm, and even more preferably between about 300 nm to about 500 nm. Irradiation is administered continuously or as pulses for hours, minutes or seconds, and preferably for the shortest amount of time possible to minimize any risk of damage to the substrate and for convenience. Radiation may be administered for less than about two hours, preferably less for than about one hour, more preferably for less than about ten minutes, and still more preferably for less than about one minute. Visible, UV and IR radiation are also preferred as all three of these forms of radiation as they do not require specialty equipment and can be commercially obtained. The intensity of the light over a given area should be sufficiently powerful as to minimize exposure time in order to achieve photocleavage. Exposure time should be minimal in order to reduce undesirable effects.

Solid supports may consist of many materials, limited primarily by capacity for derivatization to attach any of a number of chemically reactive groups and compatibility immobilization conditions. In some embodiments, solid supports include agarose and magnetic beads functionalized with bioorthogonal groups.

One common method for detection and isolation of biochemical and biological materials using bioorthogonal ligation is based on the functionalization of a solid surface (such as agarose resin or magnetic beads) with a bioorthogonal group from a bioorthogonal pair. A solution containing the corresponding bioorthogonal moiety conjugated to a target is then applied to the functionalized solid phase and the bioorthogonal reaction takes place. This reaction can be catalyzed or uncatalyzed, depending on the bioorthogonal pairs chosen. Any macromolecules which have been labeled with a bioorthogonal group can be detected and isolated using these methods.

In some embodiments, reagents according to the invention have the formula:

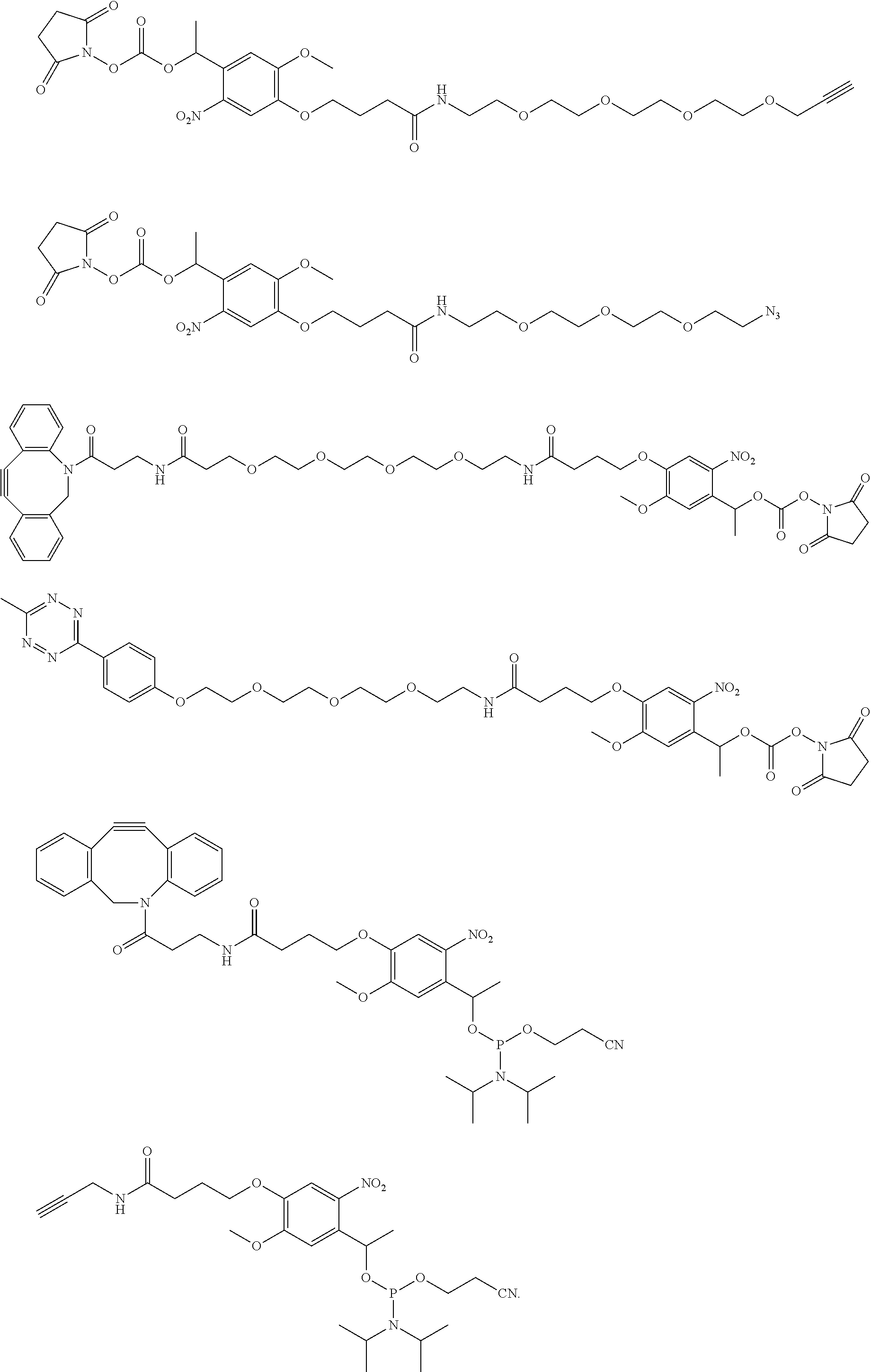

Another embodiment of the invention is directed to conjugates comprising a photocleavable bioreactive agent coupled to a substrate wherein said agent comprises a bioorthogonal moiety bonded to a photoreactive moiety, wherein said conjugate can be selectively cleaved with electromagnetic radiation to release said substrate. Suitable substrates which can be coupled to the bioreactive agent include proteins, peptides, amino acids, amino acid analogs, nucleic acids, nucleosides, nucleotides, lipids, vesicles, detergent micells, cells, virus particles, fatty acids, saccharides, polysaccharides, inorganic molecules, metals, and derivatives and combinations thereof. Substrates may be pharmaceutical agents such as cytokines, immune system modulators, agents of the hematopoietic system, chemotherapeutic agents, radio-isotopes, antigens, anti-neoplastic agents, recombinant proteins, enzymes, PCR products, receptors, hormones, vaccines, haptens, toxins, antibiotics, nascent proteins, cells, synthetic pharmaceuticals and derivatives and combinations thereof.

Another embodiment of the invention is directed to conjugates which are pharmaceutical compositions. Compositions must be safe and nontoxic and can be administered to patients such as humans and other mammals. Composition may be mixed with a pharmaceutically acceptable carrier such as water, oils, lipids, saccharides, polysaccharides, glycerols, collagens and combinations thereof and administered to patients.

Pharmaceutical compositions with photo-releasable substrates are useful, for example, for delivery of pharmaceutical agents which have short half-lives. Such agents cannot be administered through current means without being subject to inactivation before having an effect. Pharmaceutical agents in the form of conjugates, covalently bound to bioreactive agents, are more stable than isolated agents. After general administration of the composition to the patient, the site to be treated is exposed to appropriate radiation releasing substrate which produces an immediate positive response in a patient. Uncoupling from the bioreactive agent at the point of maximal biological effect is an advantage unavailable using current administration or stabilization procedures. In an analogous fashion, other areas of the patient's body may be protected from the biological effect of the pharmaceutical agent. Consequently, using these conjugates, site-directed and site-specific delivery of a pharmaceutical agent is possible.

In yet another embodiment, the present inventions relates to a process for the isolation of biomolecules, comprising
1. labeling a biomolecule with a compound of general Formula I to produce a conjugate, wherein said conjugate is a bioorthogonal moiety covalently linked to a biomolecule through a photolabile linker;
2. reacting said conjugate with solid support functionalized with complementary bioorthgonal moiety; and
3. release of captured biomolecules by exposing said solid support containing biomolecules to radiation, preferably above 300 nm.

Briefly, a conjugate is created by coupling a bioreactive agent to a substrate by a covalent bond, which is selectively cleavable with electromagnetic radiation, wherein the bioreactive agent is comprised of a bioorthogolal moiety bonded to a photocleavable moiety. The conjugate is contacted to the heterologous mixture to couple a substrate to one or more targets. The coupled conjugate is separated from the mixture and treated with electromagnetic radiation to release the substrate and the targets isolated. This method can be used to isolate targets such as immune system modulators, cytokines, agents of the hematopoietic system, proteins, hormones, gene products, antigens, cells, toxins, bacteria, membrane vesicles, virus particles, and combinations thereof from heterologous mixtures such as biological samples, proteinaceous compositions, nucleic acids, biomass, immortalized cell cultures, primary cell cultures, vesicles, animal models, mammals, cellular and cell membrane extracts, cells in vivo and combinations thereof.

One of the preferred embodiments of the invention relates to the detection or isolation of protein using bioorthogonal photocleavable reagents. In one application of this embodiment, bioorthogonal photocleavable reagent is reacted with a protein through the formation of covalent bonds with specific chemicals groups of the protein forming a conjugate. The protein may be either the target to be isolated or detected or a probe for the target protein such as an antibody. The target protein can then be isolated using solid support modified with a complementary bioorthogonal group.

The choice of a particular bioreactive agent depends on which molecular groups of the substrate are to be derivatized. For example, reaction of bioorthogonal photocleavable NHS-ester with a protein results in formation of a covalent bond with primary amino groups such as lysine residues or the NH₂ group at the N-terminal of a protein. Normally, a number of lysine residues are exposed on the surface of a protein and available for such reaction. Alternatively, several other bioorthogonal photocleavable reagents can be used which react with hydroxyl groups (—OH) present in tyrosine, threonine and serine residues, carboxyl groups (—COOH) present in aspartate and glutamate residues, and sulfhydryl groups (—SH) present in cysteine residues. Thus, a wide variety of groups are available which are likely to be on the surface of a target protein.

Another application of this embodiment is directed to the use of bioorthogonal photocleavable reagents to isolate nascent proteins that can be created from in vitro or in vivo protein synthesis. Basically, one of amino acids is replaced with a non-canonical containing amino acid with unique chemical functionality or bioorthogonal functional group such as an azide. In this method, metabolic labeling of newly synthesized proteins with, for example, azidohomoalanine (AHA) endows them with the unique chemical functionality of the azide group, which is absent in living systems (Dieterich, D. C., et al (2007) Labeling, detection and identification of newly synthesized proteomes with bioorthogonal non-canonical amino-acid tagging Nat Protoc. 2(3):532-40). In the subsequent click chemistry reaction with terminal alkyne or strained cyclooctyne covalently bound to a solid support through a photocleavable moiety, the newly synthesized proteins are covalently captured on the solid support. Nascent proteins are separated and isolated from the other components of synthesis. Photocleavage of photoliable alkyne or cyclooctyne-solid support from the nascent protein generates a pure nascent protein.

In yet another embodiment of the invention, non-canonical amino acid is incorporated into a nascent protein involving misaminoacylation of tRNA. Normally, a species of tRNA is charged by a single, cognate native amino acid. This selective charging, termed here enzymatic aminoacylation, is accomplished by enzymes called aminoacyl-tRNA synthetases and requires that the amino acid to be charged to a tRNA molecule be structurally similar to a native amino acid. Chemical misaminoacylation can be used to charge a tRNA with a non-native amino acid such as azide- or alkyne-containing amino acids.

In another embodiment, bioorthogonal photocleavable reagents are coupled to antibody. The use of bioorthogonal photocleavable reagents provides a means for recovering target molecule and the antibody for subsequent use. Release of a protein from a binding complex can be performed subsequent to the release of the binding complex from the solid support that is immobilized by complementary bioorthogonal groups. This is an advantage since it enables the release to be performed under well controlled conditions. For example, elution of a target protein from an affinity column often requires changes in buffer and/or use of a competitive agent such as an epitope, which competes for an antibody binding site. This can require long exposure of the protein to damaging conditions or the need for increased amounts of the competitive agent, which can be prohibitively expensive. In contrast, once the protein complex is removed from the solid support by photocleavage, the complex can be separated more conveniently. In the case where antibodies are used as the substrate, an additional advantage of the invention is that the antibody can also be recovered in an unaltered and purified form.

In yet another embodiment of the invention, bioorthogonal photocleavable reagents can be incorporated into antibodies or other proteins or macromolecules, and the complementary bioorthogonal group can be incorporated into another macromolecule such as protein or DNA, which can serve as hybridization probes and can also be used advantageously for sequential multiple detection of targets. Two separately labeled macromolecules are mixed together to spontaneously form a conjugate. As in the case of conventional crosslinking, two macromolecules are covalently crosslinked. However, in contrast to conventional crosslinking, the photocleavable conjugate can be completely separated.

Another embodiment of the invention is directed to target molecules isolated by the above methods, which may be used in pharmaceutical compositions or other compositions and mixtures for industrial applications.

In another embodiment, a bioorthogonal photocleavable reagent can be incorporated into DNA, RNA or PNA through chemical synthesis, PCR, DNA/RNA polymerases or any other method of chemical or enzymatic nucleotide synthesis. Once the bioorthogonal photocleavable reagent is incorporated, the target DNA or RNA can then be captured by bioorthogonal ligation with functionalized agarose or magnetic beads. The DNA and RNA containing the bioorthogonal photocleavable reagent will be retained on the solid support while other molecules will be washed away. Target sequences can then be released simply by irradiation of the sample.

Incorporation of bioorthogonal photocleavable reagents into oligonucleotides can occur through a number of methods and relies simply on providing nucleotides which have been derivatized with bioorthogonal photocleavable reagents.

Synthetic oligonucleotides of predetermined or random sequences have a variety of uses as, for example, primers, hybridization probes and antisense sequences. The synthesis of DNA and RNA oligonucleotides utilizes phosphoramidite chemistry and is routinely performed on an automated synthesizer with the growing nucleic acid chains attached to a solid support such as CPG (controlled pore glass). In this manner, phosphoramidites and other reagents can be added in excess and removed by filtration. The synthesis cycle comprises four reactions. First, acid labile trityl groups are removed from the 5′—OH groups. Second, phosphoramidites are coupled to the 5′-OH. Third, unreacted 5′—OH groups are protected by capping with acetyl groups. Finally, internucleotide linkage is converted from phosphite to phosphotriester by oxidation. This cycle is repeated until the desired sequence is obtained after which, oligonucleotide is cleaved from solid support and purified using, for example, gel electrophoresis and HPLC.

It is generally considered that coupling efficiency for each step in solid phase oligonucleotide synthesis is as much as 97-99%. Unreacted molecules are eliminated at each step by capping with acetyl groups preventing the formation of undesired sequences. Crude oligonucleotide contains, besides the full length sequence, numerous shorter sequences called the failure sequences. Purification of such a complex mixture is difficult especially when it comes to isolation of full-length sequence from slightly shorter failure sequences, such as those lacking only 1 or 2 bases. This problem becomes even more difficult when synthesizing long sequences of DNA or RNA where coupling efficiencies are lower, resulting in more failure sequences. 5′-bioorthogonal photocleavable phosphoramidites can be used to selectively label full-length oligonucleotides at their 5′-end during solid-support synthesis on automated nucleic acid synthesizers.

Bioorthogonal photocleavable phosphoramidites can contain the bioorthogonal and photocleavable moieties linked to a phosphoramidite functionality through a spacer arm allowing for efficient bioorthogonal ligation. This reagent selectively reacts with the 5′—OH group on a sugar ring and can be used on automated synthesizers. In addition, photocleavage of 5′-photocleavable nucleic acid results in formation of 5′-phosphorylated sequences. 5′-phosphorylated oligonucleotides are generally required for most downstream applications.

In preferred embodiments of the invention, 5′ bioorthogonal photocleavable reagents are incorporated into an oligonucleotide during solid phase synthesis. Briefly, after the target sequence is achieved using standard synthesis procedures, an additional detritylation is performed, and the bioorthogonal photocleavable phosphoramidite is added to the reaction vessel in excess. The excess bioorthogonal photocleavable phosphoramidite is removed and the oligonucleotide is cleaved from the resin. Unlike photocleavable biotin phosphoramidites, there is no need for additional detritylation since bioorthogonal moieties do not generally need to be protected during oligonucleotide synthesis. After cleavage from the synthesis resin, the crude oligonucleotide solution is added to a solid support or solid suspension such as magnetic beads which have been functionalized with the bioorthogonal group which is complimentary to the bioorthogonal group on the oligonucleotide. After bioorthogonal ligation, the solution is separated from the solid support followed by washing. Once failure sequences and other non-target molecules are removed, the pure oligonucleotide can be photocleaved and collected or left attached to the resin for additional downstream applications.

The invention contains a number of chemical moieties for conjugation to targets. In one case, an NHS-ester functionality introduced in bioorthogonal photocleavable reagents, which is reactive toward aliphatic amino groups, are present in proteins. Another example is a phosphoramidite moiety, which is highly reactive and can selectively react with hydroxyl groups of nucleic acids. These derivatives can be chemically linked to a variety of macromolecules and molecular components including amino acids, nucleotides, proteins and polypeptides, nucleic acids (DNA, RNA,), lipids, hormones and molecules which function as ligands for receptors.

Choice of photolabile group, spacer arm and the bioorthogonal moiety depends on the target substrate including amino acids, proteins, antibodies, nucleotides, DNA or RNA, lipids, carbohydrates and cells to which the bioorthogonal photocleavable reagents to be attached. It also depends on the exact conditions for photocleavage and the desired interaction between the bioorthogonal moiety and the corresponding pair.

Oftentimes, it is useful to include a spacer moiety between the photoreactive moiety and the bioorthogonal group. The presence of the spacer can be advantageous sterically for ligation to proceed efficiently. The spacer moiety may comprise a branched or straight chain hydrocarbon, a polymeric carbohydrate, or a derivative or combination thereof. A common spacer group is a polymer of polyethylene glycol (PEG).

Another embodiment of the invention is directed to photocleavable conjugates comprising bioreactive agents photocleavably coupled to substrates. Conjugates have the property that they can be selectively cleaved with electromagnetic radiation to release the substrate. Substrates are those chemicals, macromolecules, cells and other substances which are or can be used to detect and isolate targets. Substrates that are selectively cleaved from conjugates may be modified by photocleavage, containing the bioorthogonal group, containing the products of the bioorthogonal reaction, but still functionally active, or may be released from the conjugate completely unmodified by photocleavage.

A substrate can represent any molecule, macromolecule or cell that can be attached to a bioreactive agent. These substrates could be proteins, peptides, amino acids, amino acid analogs, nucleic acids, nucleosides, nucleotides, lipids, vesicles, detergent micells, cells, virus particles, fatty acids, saccharides, polysaccharides, inorganic molecules and metals. Substrates may also comprise derivatives and combinations of these substances such as fusion proteins, protein-carbohydrate complexes and organo-metallic compounds. Substrates may also be recombinant proteins, chemotherapeutic agents, radio-isotopes, antigens, anti-neoplastic agents, enzymes, PCR products, receptors, hormones, vaccines, haptens, toxins, antibiotics, nascent proteins, synthetic pharmaceuticals and derivatives and combinations thereof.

Substrates themselves may be targets or part of the targets such as an amino acid in the synthesis of nascent polypeptide chains wherein substrates may be an amino acid or an amino acid derivative incorporated into the polypeptide chain. Given substrates may also include nucleotides or nucleotide derivatives as precursors in the synthesis of nucleic acids. Precursors or modified precursors useful in creating synthetic oligonucleotide conjugates may contain phosphoramidites or derivatives of dATP, dCTP, dTTP and dGTP, and also ATP, CTP, UTP and GTP. The nucleic acid-conjugates can be used in PCR technologies, antisense therapy, and prophylactic and diagnostic applications.

The process of photocleavage should preferably not damage a released substrate or impair substrate activity. Proteins, nucleic acids and other protective groups used in peptide and nucleic acid chemistry are known to be stable to most wavelengths of radiation above 300 nm. The yield and exposure time necessary for release of substrate photo-release are strongly dependent on the structure of the photoreactive moiety. In the case of unsubstituted 2-nitrobenzyl PCB derivatives, the yield of photolysis and recovery of the substrate are significantly decreased by the formation of side products which act as internal light filters and are capable of reacting with amino groups of the substrate. In this case, illumination times vary from about 1 minute to about 24 hours, preferably less than 4 hours, more preferably less than two hours, and even more preferably less than one hour, and yields are between about 1% to about 95% (V. N. R. Pillai, Synthesis 1, 1980). In the case of alpha-substituted 2-nitrobenzyl derivatives (methyl, phenyl), there is a considerable increase in rate of photo-removal as well as yield of the released substrate (J. E. Baldwin et al., Tetrahedron 46:6879, 1990; J. Nargeot et al., Proc. Natl. Acad. Sci. USA 80:2395, 1983).

The determination of which bioreactive agent to use depends strongly on the molecular entity of the substrate which is to be modified. For example, the reaction of a photocleavable bioorthogonal (such as alkyne or DBCO) NHS-ester with a protein results in the formation of a covalent bond with primary amino groups such as at the ε-position of lysine residues or the α-NH₂ group at the N-terminal of a protein. In most proteins, a number of lysine residues are surface-exposed and available for such reaction. Alternatively, several other photocleavable bioorthogonal reagents can be used which react with hydroxyl groups (—OH) present in tyrosine, threonine and serine residues, carboxyl groups (—COOH) present in aspartate and glutamate residues, and sulfhydryl groups (—SH) present in cysteine residues. These represent a wide variety of groups that are available which are likely to be on the surface of a target protein.

Attachment of photocleavable bioorthogonal reagent to molecules which bind proteins such as receptor ligands, hormones, antibodies, nucleic acids, and proteins that bind glycoproteins can also be accomplished because of the wide variety of reactive groups available for photocleavable bioorthogonals. For example, photocleavable bioorthogonal reagent can be conveniently linked to antibodies which are directed against a particular protein. Alternatively, photocleavable bioorthogonal reagents can be linked to DNA and RNA with sequences complimentary to a target RNA or DNA or proteins which bind nucleic acids.

Conjugates of the invention may be attached to a solid support via the bioorthogonal group, the substrate or any other chemical group of the structure. The solid support may comprise constructs of glass, ceramic, plastic, metal or a combination of these substances. Useful structures and constructs include plastic structures such as microtiter plate wells or the surface of sticks, paddles, beads or microbeads, alloy and inorganic surfaces such as semiconductors, two and three dimensional hybridization and binding chips, and magnetic beads, chromatography matrix materials and combinations of these materials.

Another embodiment of the invention is directed to a method for isolating targets from a heterogeneous mixture. Bioreactive agents are contacted with the mixture to react with a target forming the conjugate. Alternatively, conjugates can be contacted with the heterologous mixture to couple a substrate within the conjugate to one or more targets. Conjugates can be separated from the mixture by any currently available techniques, such as magnetic separations, separations based on side and centrifugal force, among others.

Procedures such as chemical or physical separation of components of the mixture, electrophoresis, electroelution, sedimentation, centrifugation, filtration, magnetic separation, chemical extraction, affinity separation methods such as affinity chromatography or another chromatographic procedure such as ion-exchange, gradient separation, HPLC or FPLC, and combinations of these techniques are well-known and allow for a rapid isolation with a high efficiency of recovery (e.g. M. Wilchek et al., Methods Enzymol. 184, 1990; M. Wilchek et al., Anal. Biochem. 171:1, 1988). After separation or isolation, targets can be easily quantitated using available methods such as optical absorbance or transmission (e.g. nucleic acid, proteins, lipids) or the Bradford (M. Bradford, Anal. Biochem. 72:248, 1976) or Lowry (O. Lowry et al., J. Biol. Chem. 193:265, 1951) assays (e.g., proteins), both of which are commercially available. After separation, coupled conjugates are treated with electromagnetic radiation to release the substrate. The substrate targets can then be separated from the released bioreactive agent, if desired, to obtain substantially or completely pure targets.

Detection and isolation are determined by the ability of the bioreactive agent to bind the substrate. For example, nucleic acids can be base-paired to complementary nucleic acids, to nucleic acid binding proteins or to chemical moieties, which react specifically with chemical moieties found on nucleic acids. Proteins can be bound with monoclonal or polyclonal antibodies or antibody fragments specific to those proteins, or chemical moieties which react specifically with chemical moieties found on the proteins of interest.

The heterologous mixture which contains the target may be a biological sample, any proteinaceous composition such as a cellular or cell-free extract, nucleic acid containing compositions, a biomass containing, for example, vegetative or microbial material, a cell culture of primary or immortalized cells. The substrate can also be proteins, peptides, amino acids, amino acid analogs, nucleosides, nucleotides, lipids, vesicles, detergent micells, fatty acids, saccharides, polysaccharides, inorganic molecules, metals and derivatives and combinations thereof.

In an application of this method, the substrate may be an integral component of the target such as a nucleotide in the detection and isolation of nascent nucleic acids or an amino acid in the detection and isolation of nascent proteins. The substrate is incorporated into the target by chemical or enzymatic techniques and detected and isolated by the presence of the detectable moiety.

In another application of the preferred embodiment, a photocleavable bioorthogonal reagent can be incorporated into a DNA (deoxyribonucleic acid), RNA (ribonucleic acid) or PNA (polynucleic amide) molecule produced by chemical synthesis, PCR, nick translation or DNA or RNA polymerases or by any other production method. Target DNA or RNA can then be isolated by using any bioorthogonal ligation methods. Isolation is accomplished, for example, using commercially available magnetic beads functionalized with azides. Ligation will occur between the beads and the DNA and/or RNA containing the photocleavable bioorthogonal reagent moiety, whereas all other molecules are washed away. The photocleavable bioorthogonal linkage is then removed from the target nucleic acid by illumination.

The incorporation of photocleavable bioorthogonal reagent into nucleic acids such as DNA and RNA involves the synthesis and use of compounds that are formed from the derivatization of nucleotides with photocleavable bioorthogonal groups.

Bioorthogonal ligation is currently used in the field of molecular biology and biomedicine as a means for efficiently isolating the products of DNA and RNA synthesis as well as for detection of specific sequences in nucleic acids. Isolation normally involves the attachment or incorporation of a “click” group into the DNA or RNA followed by ligation of the oligonucleotide onto a solid support. For example, this method can be used to probe an RNA sequence of interest using a complimentary DNA or RNA sequence containing a bioorthogonal group which is incorporated during PCR. Once immobilized, however, it is not possible to release the probe from the solid support, meaning single stranded nucleotides must be denatured for release. The utilization of photolabile linkers in the isolation or detection of nucleic acids allows for the release of the probe and target in a single step. This means that single stranded DNA or DNA duplex can be release in a pure form in a single step from a bioorthogonal functionalized solid support.

In a preferred embodiment of this invention, the isolation of nucleic acids occurs in three steps. First, a photocleavable bioorthogonal derivative is attached to a nucleic acid molecule by enzymatic or chemical means or, alternatively, by incorporation of a photocleavable bioorthogonal nucleotide into a nucleic acid by enzymatic or chemical means. The choice of a particular photocleavable bioorthogonal group depends on which molecular groups are to be derivatized on the nucleic acid. For example, attachment of a photocleavable bioorthogonal group to a nucleic acid can be accomplished by forming a covalent bond with the aromatic amine, sugar hydroxyls or phosphate groups. Photocleavable bioorthogonal reagent can also be incorporated into oligonucleotides through enzymatic means. Next, the nucleic acid molecule is separated through the selective ligation with the corresponding bioorthogonal partner, which can be functionalized onto a solid support. For example, nucleic acid molecules containing photocleavable bioorthogonal reagent, where the bioorthogonal group is a terminal alkyne, are ligated to magnetic beads functionalized with azide in a copper catalyzed reaction. The remaining unbound biomolecules are then washed to remove other reactants, buffer and salts. Since the oligonucleotide is covalently linked to the solid support, binding and washing conditions can be as stringent as desired, providing the conditions are compatible with the oligonucleotide. Finally, the photocleavable bioorthogonal reagent is detached from the nucleic acid by illumination at a wavelength which causes the photocleavable covalent linkage to be broken. Separation of the oligonucleotide from the PCBO results in a pure oligonucleotide.

The following examples are offered to illustrate various embodiments of the invention but should not be viewed as limiting the scope of the invention.

### Examples

### Synthesis of Photolabile Compounds

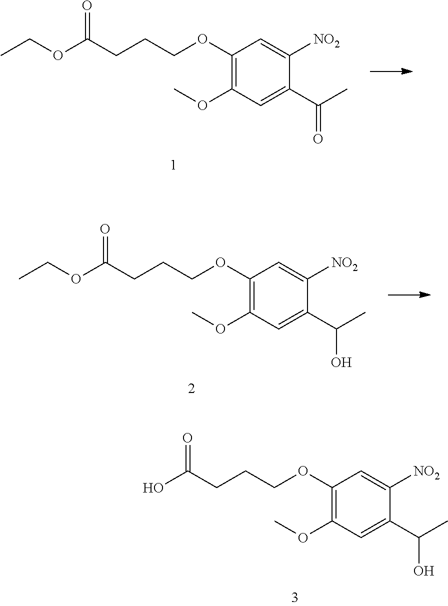

Sodium borohydride (9.31 g, 369 mmol) was added to a suspension of ethyl 4-(4-acetyl-2-methoxy-5-nitrophenoxy)butanoate (1) (30 g, 92 mmol, prepared according to WO 2013/057186 A1) in MeOH (300 ml) in several portions. The progress of the reaction was following by TLC (EtOAc:Hex 1:1) analysis. Upon completion, the reaction was quenched by aqueous NH₄Cl (ca. 20 mL), partially concentrated, diluted with water (ca. 100 mL), and EtOAc (ca. 200 mL), the organic layer was separated, washed with water, brine, dried over Na₂SO₄ and concentrated to provide 24.1 g of ethyl 4-(4-(1-hydroxyethyl)-2-methoxy-5-nitrophenoxy)butanoate (2) as a slightly yellow solid.

A solution of lithium hydroxide hydride (2.341 g, 98 mmol) in water (ca. 50 mL) was added to a solution of crude ethyl 4-(4-(1-hydroxyethyl)-2-methoxy-5-nitrophenoxy)butanoate (2) (8 g, 24.44 mmol) in THF (50 mL) and MeOH (50 mL) at room temperature, and the reaction mixture was stirred for ca. 2 hours at this temperature. According to TLC analysis (EtOAc:Hex 1:1), all product was consumed, the reaction mixture was concentrated, and the product was crashed by addition of 5% HCl. The crude product was filtered, and dried on pump to provide 7.1 g of crude 4-(4-(1-hydroxyethyl)-2-methoxy-5-nitrophenoxy)butanoic acid (3) product as grey solid.

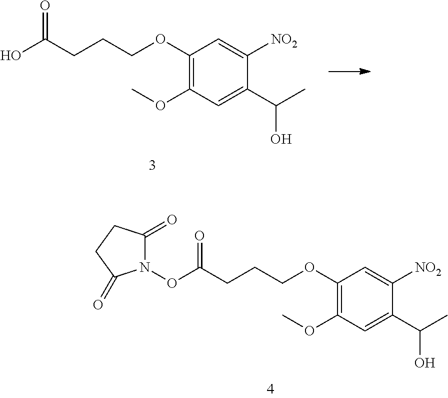 [SMILES: COc1cc(C(C)O)c([N+](=O)[O-])cc1OCCCC(=O)O ; COc1cc(C(C)O)c([N+](=O)[O-])cc1OCCCC(=O)ON1C(=O)CCC1=O]

EDC (14.01 g, 73.1 mmol) was added to a suspension of 4-(4-(1-hydroxyethyl)-2-methoxy-5-nitrophenoxy)butanoic acid (3) (16.2 g, 54.1 mmol), and NHS (7.16 g, 62.3 mmol) in DMF (150 ml) at room temperature, and the reaction mixture was stirred for ca. 60 min. Upon completion (TLC, DCM:MeOH 20:1), the reaction mixture was concentrated, re-dissolved in DCM, washed with 5% HCl, dried over Na₂SO₄, and concentrated to provide 15.2 g of crude product (2,5-dioxopyrrolidin-1-yl 4-(4-(1-hydroxyethyl)-2-methoxy-5-nitrophenoxy)butanoate (4)) that may be used without any further purification.

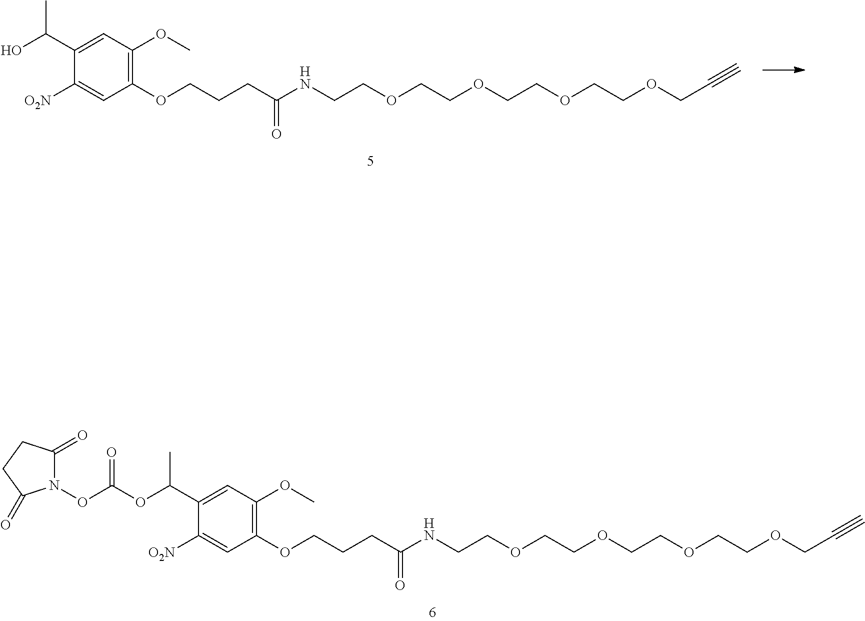 [SMILES: C#CCOCCOCCOCCOCCNC(=O)CCCOc1cc([N+](=O)[O-])c(C(C)O)cc1OC ; C#CCOCCOCCOCCOCCNC(=O)CCCOc1cc([N+](=O)[O-])c(C(C)OC(=O)ON2C(=O)CCC2=O)cc1OC]

Alkyne-PEG4-amine (1.682 g, 7.27 mmol) was added to a solution of 2,5-dioxopyrrolidin-1-yl 4-(4-(1-hydroxyethyl)-2-methoxy-5-nitrophenoxy)butanoate (4) (2.5 g, 6.32 mmol) in DCM (25 ml) at room temperature. The reaction mixture was stirred for ca. 30 min. According to TLC analysis, all NHS ester was consumed. The reaction mixture was transferred into a separatory funnel, washed with 5% HCl, dried over Na₂SO₄ and concentrated. The crude product was purified on silica gel (5% MeOH in DCM) to provide the Alkyne-PC-OH 5 as a yellow oil.

DSC (1.805 g, 7.05 mmol) was added to a solution of Alkyne-PC-OH 5 (2.33 g, 4.55 mmol), triethylamine (0.920 g, 9.09 mmol), and DMAP (0.056 g, 0.455 mmol) in several portions over ca. 30 min, and the reaction mixture was stirred for additional 30 min at room temperature. The progress of the reaction was followed by TLC and/or HPLC. Upon completion, the reaction mixture was transferred into a separatory funnel, washed with NaHCO₃, 5% HCl, dried over Na₂SO₄ and concentrated. The crude product was purified on silica gel to provide NHS ester 6 as a slightly yellow oil.

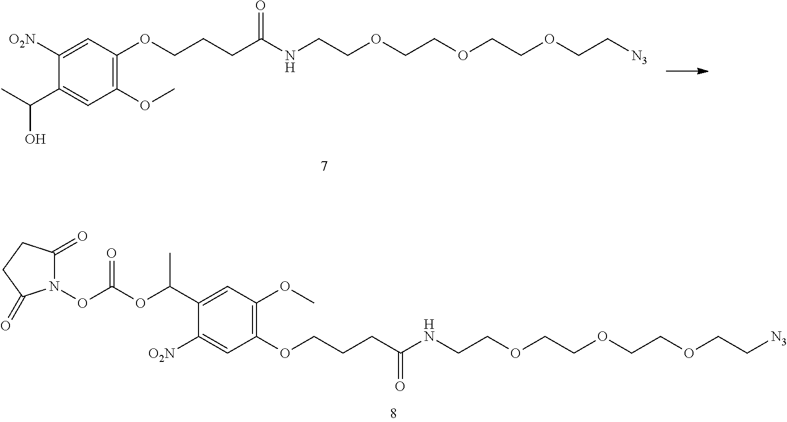 [SMILES: COc1cc(C(C)O)c([N+](=O)[O-])cc1OCCCC(=O)NCCOCCOCCOCCN=[N+]=[N-] ; COc1cc(C(C)OC(=O)ON2C(=O)CCC2=O)c([N+](=O)[O-])cc1OCCCC(=O)NCCOCCOCCOCCN=[N+]=[N-]]

A solution of Azido-PEG3-amine (1.678 g, 8.73 mmol) in ca. 5 mL of DCM was added to a suspension of 4 (3 g, 7.59 mmol) in DCM (50 ml) at room temperature, and the reaction mixture was stirred for ca. 60 min. The progress of the reaction was followed by TLC and/or HPLC. Upon completion, the reaction mixture was transferred into a separatory funnel, washed with 5% HCl, dried over Na₂SO₄ and concentrated. The crude product was purified on silica gel to provide crude compound 7 as a yellow oil.

DSC (1.218 g, 4.76 mmol) was added to a solution of crude compound 7 (1.76 g, 3.52 mmol), triethylamine (1.019 ml, 7.05 mmol), and DMAP (0.043 g, 0.352 mmol) in several portions over ca. 30 min, and the reaction mixture was stirred for an additional 30 min at room temperature. The progress of the reaction was followed by TLC and/or HPLC. Upon completion, the reaction mixture was transferred into a separatory funnel, washed with NaHCO₃, 5% HCl, dried over Na₂SO₄ and concentrated. The crude product was purified on silica gel to provide compound 8 as a slightly yellow oil.

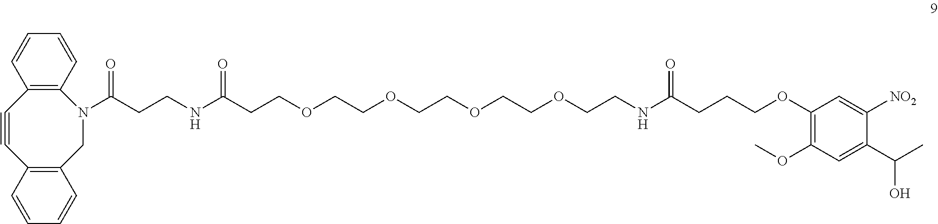 [SMILES: COc1cc(C(C)O)c([N+](=O)[O-])cc1OCCCC(=O)NCCOCCOCCOCCOCCC(=O)NCCC(=O)N1Cc2ccccc2C#Cc2ccccc21]

DBCO-PEG4-Amine (4.5 g, 8.59 mmol) (Click Chemistry Tools, Scottsdale, Ariz.) was added to a solution of 2,5-dioxopyrrolidin-1-yl 4-(4-(1-hydroxyethyl)-2-methoxy-5-nitrophenoxy)butanoate (4) (3.40 g, 8.59 mmol) at room temperature, and the reaction mixture was stirred for ca. 2 hours. The progress of the reaction was followed by TLC and/or HPLC. Upon completion, the reaction mixture was transferred into a separatory funnel, washed with 5% HCl, dried over Na₂SO₄ and concentrated. The crude was purified on silica gel to provide the compound 9 as a yellow oil.

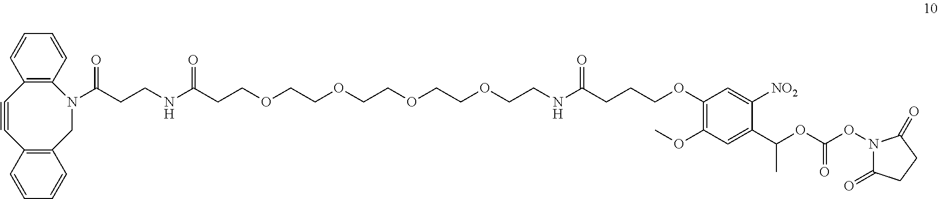 [SMILES: COc1cc(C(C)OC(=O)ON2C(=O)CCC2=O)c([N+](=O)[O-])cc1OCCCC(=O)NCCOCCOCCOCCOCCC(=O)NCCC(=O)N1Cc2ccccc2C#Cc2ccccc21]

DCS (2.072 g, 8.09 mmol) was added in several portions to a solution of product 9 (4.2 g, 5.22 mmol), triethylamine (1.056 g, 10.44 mmol), and N,N-dimethylpyridin-4-amine (0.064 g, 0.522 mmol) at room temperature. An additional amount of DCS was added over ca. 2 hours until all of product 9 was converted into NHS ester. Upon completion, the reaction mixture was transferred into a separatory funnel, washed with 5% HCl, dried over Na₂SO₄ and concentrated. The crude product was purified on silica gel to provide the DBCO-PC-NHS Ester 10 as a yellow solid.

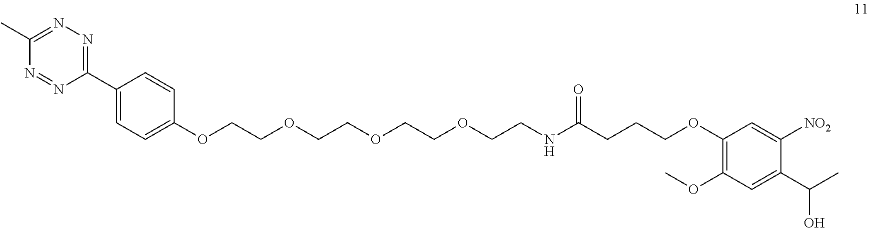 [SMILES: COc1cc(C(C)O)c([N+](=O)[O-])cc1OCCCC(=O)NCCOCCOCCOCCOc1ccc(-c2nnc(C)nn2)cc1]

Compound 4 (2 g, 5.06 mmol) was added to a solution of Methyltetrazine-PEG4-Amine (2.023 g, 5.06 mmol) (Click Chemistry Tools, Scottsdale, Ariz.) and triethylamine (1.536 g, 15.18 mmol) in DCM (25 mL) at room temperature and the reaction mixture was stirred for ca. 2 hours. The progress of the reaction was followed by TLC and/or HPLC. Upon completion, the reaction mixture was transferred into a separatory funnel, washed with 5% HCl, dried over Na₂SO₄ and concentrated. The crude product was purified on silica gel to provide the crude Methyltetrazine-PC-OH 11 as a red solid. The crude product was purified on silica gel (EtOAc:MeOH).

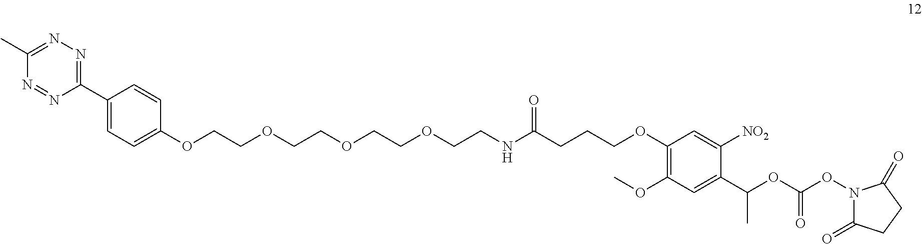 [SMILES: COc1cc(C(C)OC(=O)ON2C(=O)CCC2=O)c([N+](=O)[O-])cc1OCCCC(=O)NCCOCCOCCOCCOc1ccc(-c2nnc(C)nn2)cc1]

DCS (1.75 g, 6.83 mmol) was added in several portions to a solution of Methyltetrazine-PC-OH 11 (2.2 g, 3.41 mmol), triethylamine (0.69 g, 6.83 mmol), and N,N-dimethylpyridin-4-amine (0.042 g, 0.341 mmol) at room temperature. An additional amount of DCS was added over ca. 2 hours until all Methyltetrazine-PC-OH 11 was converted into NHS ester. Upon completion, the reaction mixture was transferred into a separatory funnel, washed with 5% HCl, dried over Na₂SO₄ and concentrated. The crude product was purified on silica gel to provide Methyltetrazine-PC-NHS Ester 12 as a red solid.

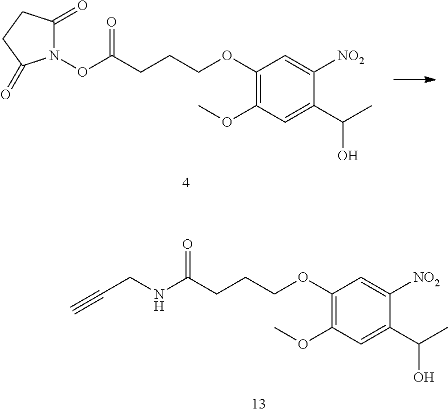

Propargylamine (0.278 g, 5.05 mmol) was added to a solution of 2,5-dioxopyrrolidin-1-yl 4-(4-(1-hydroxyethyl)-2-methoxy-5-nitrophenoxy)butanoate (2 g, 5.05 mmol) in DCM (25 ml) at room temperature. The reaction mixture was stirred for ca. 30 min. According to TLC analysis, all NHS ester was consumed. The reaction mixture was transferred into a separatory funnel, washed with 5% HCl, dried over Na₂SO₄ and concentrated. The crude product was purified on silica gel to provide 4-(4-(1-hydroxyethyl)-2-methoxy-5-nitrophenoxy)-N-(prop-2-yn-1-yl)butanamide (13) as a yellow solid.

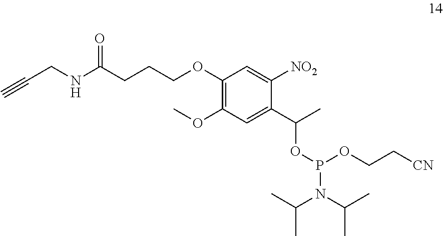 [SMILES: C#CCNC(=O)CCCOc1cc([N+](=O)[O-])c(C(C)OP(OCCC#N)N(C(C)C)C(C)C)cc1OC]

2-Cyanoethyl N,N-diisopropylchlorophosphoramidite (0.728 g, 3.08 mmol) was added to a suspension of 4-(4-(1-hydroxyethyl)-2-methoxy-5-nitrophenoxy)-N-(prop-2-yn-1-yl)butanamide (13) (0.9 g, 2.68 mmol) and DIEA (0.692 g, 5.35 mmol) with ice-bath cooling, and the reaction mixture was stirred for ca. 30 min. According to TLC analysis (EtOAc), all substrate was consumed. The reaction mixture was transferred into a separatory funnel, washed with NaHCO₃, dried over Na₂SO₄ and concentrated. The crude product was purified on silica gel to provide the product 14 as a yellow solid.

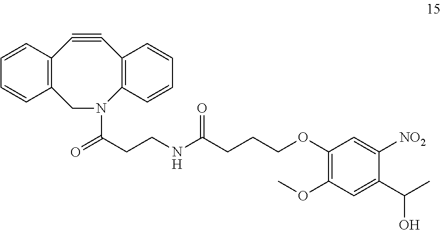 [SMILES: COc1cc(C(C)O)c([N+](=O)[O-])cc1OCCCC(=O)NCCC(=O)N1Cc2ccccc2C#Cc2ccccc21]

DBCO-Amine (0.877 g, 3.17 mmol) (Click Chemistry Tools, Scottsdale, Ariz.) was added to a solution of 2,5-dioxopyrrolidin-1-yl 4-(4-(1-hydroxyethyl)-2-methoxy-5-nitrophenoxy)butanoate (4) in DCM (25 ml) at room temperature. The reaction mixture was stirred for ca. 30 min. According to TLC analysis, all NHS ester was consumed. The reaction mixture was transferred into a separatory funnel, washed with 5% HCl, dried over Na₂SO₄ and concentrated. The crude product was purified on silica gel to provide PC-DBCO-OH 15 as a yellow solid.

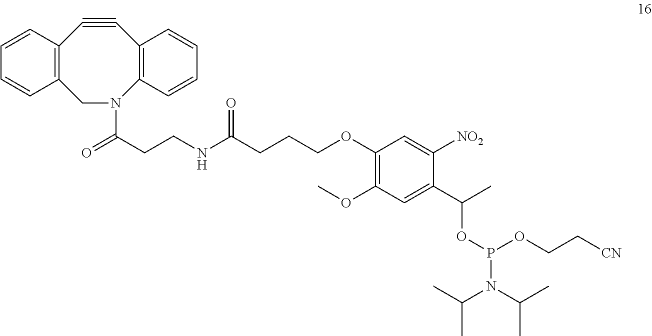 [SMILES: COc1cc(C(C)OP(OCCC#N)N(C(C)C)C(C)C)c([N+](=O)[O-])cc1OCCCC(=O)NCCC(=O)N1Cc2ccccc2C#Cc2ccccc21]

2-Cyanoethyl N,N-diisopropylchlorophosphoramidite (0.488 g, 2.06 mmol) was added to a suspension of PC-DBCO-OH 15 (1.0 g, 1.793 mmol) and DIEA (0.464 g, 3.59 mmol) with ice-bath cooling, and the reaction mixture was stirred for ca. 30 minutes at 4° C. According to TLC analysis (EtOAc), all substrate was consumed. The reaction mixture was transferred into a reparatory funnel, washed with NaHCO₃, dried over Na₂SO₄ and concentrated. The crude product was purified on silica gel to provide compound 16 as a yellow oil.

Capture and Release of β-Lactoglobulin with Compound 10

This example demonstrates the capture of labeled substrates with the compounds of the present invention and their subsequent photorelease and characterization. 1.3 μmoles of compound 10 was added to a 460 μM solution of β-lactoglobulin (325 nmoles) in PBS, pH 7.5 and allowed to react for 45 minutes. Excess of unreacted compound 10 was removed from the β-lactoglobulin solution using a desalting column according to the manufacturer's instructions. β-lactoglobulin labeled with compound 10 (35 nmoles) was added to a solution containing RNase (364 μM), thyroglobulin (7.5 μM), and gamma globulins from goat serum (5 mg/ml) in 50 mM Tris/4M Urea/2% CHAPS/500 mM NaCl, pH 8.0. This protein solution (containing compound 10 labeled-β-lactoglobulin) was added to 50 μL of azide-agarose resin and allowed to incubate overnight with constant agitation. The agarose beads were then separated from the solution by centrifugation, washed twice with 1% SDS, twice with 8M Urea and 5 times with PBS. Following the wash steps, the agarose beads containing captured β-lactoglobulin were resuspended in 500 μL of PBS and irradiated using a hand held UV-lamp for 30 min with agitation. The lamp was set to the long-wave UV setting, emitting at 365 nm. After photorelease, the beads were separated from the solution by centrifugation and resuspended in 1% SDS. This post-elution detergent wash was separated from the beads and retained. Samples were subjected to analysis by SDS-PAGE and analyzed using densiometry.

Capture and Release of β-Lactoglobulin with Compound 12

1.3 μmoles of compound 12 was added to a 460 μM solution of β-lactoglobulin (325 nmoles) in PBS, pH 7.5 and allowed to react for 45 minutes. Excess, unreacted compound 12 was removed from the β-lactoglobulin solution using a desalting column according to the manufacturer's instructions. β-lactoglobulin labeled with compound 12 (35 nmoles) was added to a solution containing RNase (364 μM), thyroglobulin (7.5 μM), and gamma globulins from goat serum (5 mg/ml) in 50 mM Tris/4M Urea/2% CHAPS/500 mM NaCl, pH 8.0. This protein solution (containing compound 12 labeled-β-lactoglobulin) was added to 50 μl of agarose-TCO resin and allowed to incubate overnight with constant agitation. The agarose beads were then separated from the solution by centrifugation, washed twice with 1% SDS, twice with 8 M Urea and 5 times with PBS. Following the wash steps, the agarose beads containing captured β-lactoglobulin were resuspended in 500 μL of PBS and irradiated using a hand held UV-lamp for 30 min with agitation. The lamp was set to the long-wave UV setting, emitting at 365 nm. After photorelease, the beads were separated from the solution by centrifugation and resuspended in 1% SDS. This post-elution detergent wash was separated from the beads and retained. Samples were subjected to analysis by SDS-PAGE and analyzed using densiometry.

### Capture and Release of Oligonucleotides

The DNA sequence 1—[NH2]GG CCG CTA CCT CTC ACC ACT CAT G was synthesized using standard solid phase procedures including a 5′ amine. A 420 μM solution of DNA sequence 1 was labeling using 40 times excess compound 8 in PBS buffer containing 1 mM EDTA. After 45 min, the DNA was precipitated by the addition of 150 mM sodium acetate and an equal volume of cold anhydrous ethanol. After centrifugation, the DNA pellet was resuspended in PBS+1 mM EDTA and added to 10 uL of DBCO-Agarose resin. The DNA-agarose suspension was allowed to mix for 16 hours with constant agitation. After incubation, the agarose beads were washed 3× with PBS+1 mM EDTA before resuspension in 500 μl of the same buffer. The DNA was released from the beads by irradiation using a hand held UV lamp emitting at 365 nm for 15 min. The solution containing the photoreleased DNA was analyzed by HPLC (mobile phase A: 50 mM TEAA; mobile phase B: acetonitrile; 0-85% B over 18 min).

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A compound of general formula:
R₁-L₁-PCL-L₂-R₂
wherein PCL is a photolabile group;
R₁ is selected from the group consisting of —O—, —OH, —SH, —NH, —NH₂, —F, —Cl, —Br, —I, O-Su (N-hydroxysuccinimidyl, sulfo-N-hydroxysuccinimidyl), —O-STP (4-sulfo-2,3,5,6,-tetrafluorophenyl), —O-TFP (2,3,5,6-pentafluorophenyl), —O-benzotriazole, benzotriazole, benzotriazole carbonate, p-nitrophenyl carbonate, benzotrizole carbonate, 2,3,5-trichlorophenyl carbonate, succinimidyl carbonate, —COCl, —SO₂Cl, —CO—CH₂—I, —COO—, —COOH, —CO—NH—NH₂, —O-phosphoramidite, —CHO, -maleimide, and pyridylsulfide;
L₁ is a non-cleavable linker or absent;
L₂ is a non-cleavable linker or absent;
R₂ is a reactive partner of a pair of orthogonally reactive moieties that can react with each other in the presence or absence of a catalyst without activation and both reactive moieties are sufficiently stable under commonly applied biomolecule labeling conditions.

2. The compound according to claim 1, wherein said photolabile group is selected from the group consisting of o-alkylated nitrophenyl, o-alkylated aryl ketones, a benzoin group, a p-hydroxyphenacyl group, a coumarinyl group, and α-substituted methylphenols.

3. The compound according to claim 1, wherein said compound is selected from the group consisting of: 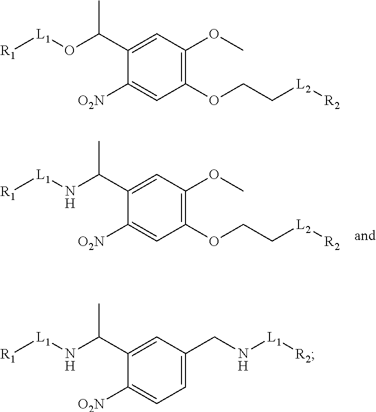
wherein R₁, R₂, L₁, and L₂ are defined in claim 1.

4. The compound according to claim 1, wherein R₁ is an amine reactive activated ester or activated carbonate capable of modifying primary amine-containing biomolecules.

5. The compound according to claim 4, wherein the amine reactive activated ester or activated carbonate is selected from the group consisting of N-hydroxysuccinimidyl, sulfo-N-hydroxysuccinimidyl, 4-sulfo-2,3,5,6,-tetrafluorophenyl, 2,3,5,6-tetrafluorophenyl, —O-benzotriazole, benzotriazole carbonate, p-nitrophenyl carbonate, benzotrizole carbonate, 2,3,5-trichlorophenyl carbonate, and succinimidyl carbonate.

6. The compound according to claim 1, wherein R₁ is a sulfhydryl reactive group capable of modifying sulfhydryl-containing biomolecules.

7. The compound according to claim 6, wherein the sulfhydryl reactive group is selected from the group consisting of maleimide, iodoacetyl, pyridildisulphide, vinylsulfone, and α,β-unsaturated carbonyl.

8. The compound according to claim 1, wherein R₁ is a phosphoramidite group.

9. The compound according to claim 1, wherein R₂ is selected from the group consisting of orthogonal reactive pairs which undergo Staudinger ligation, copper-catalyzed Huisgen 1,3-dipolar cycloaddition, strain-promoted Huisgen 1,3dipolar cycloaddition, Inverse Electron Demand Diels-Alder cycloaddition, and hydrazone or oxime bond forming reactions.

10. A kit comprising at least one compound according to claim 1.

11. A compound of general formula:
R₁-L₁-PCL-L₂-R₂
wherein PCL is a photolabile group;
R₁ is a biomolecule;
L₁ is a non-cleavable linker or absent;
L₂ is a non-cleavable linker or absent;
R₂ is a reactive moiety partner of a pair of orthogonally reactive moieties that can react with each other in the presence or absence of a catalyst without activation and both reactive moieties are sufficiently stable under commonly applied biomolecule labeling conditions.

12. The compound according to claim 11, wherein said the photoliable group is selected from o-alkylated nitrophenyl, o-alkylated aryl ketones, the benzoin group, the p-hydroxyphenacyl group, the coumarinyl group, or α-substituted methylphenols.

13. The compound according to claim 11, wherein said compound is selected from the group consisting of: 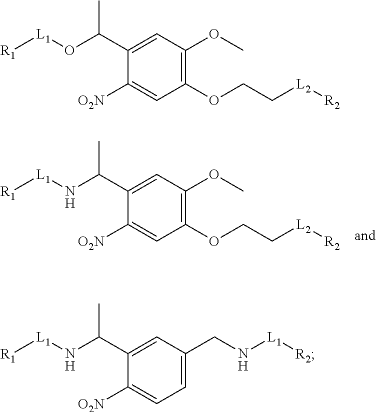
wherein R₁, R₂, L₁, and L₂ are defined in claim 11.

14. The compound according to claim 11, wherein said biomolecule is selected from the group consisting of proteins, peptides, amino acids, lipids, cells, virus particle, fatty acids, polysaccharides, and synthetic or natural, single or double strand DNA or RNA.

15. The compound according to claim 11, wherein R₂ is selected from the group consisting of orthogonal reactive pairs that can undergo Staudinger ligation, copper-catalyzed Huisgen 1,3-dipolar cycloaddition, strain-promoted Huisgen 1,3dipolar cycloaddition, Inverse Electron Demand Diels-Alder cycloaddition, and hydrazone or oxime bond forming reactions.

16. A kit comprising at least one compound according to claim 11.

17. A method, which comprises:
modifying a biomolecule with the compound of claim 1 to produce a conjugate, wherein said conjugate contains a bioorthogonal moiety covalently linked to the biomolecule through a photolabile linker;
reacting said conjugate with a solid support or another molecule functionalized with a complementary bioorthgonal moiety; and
photocleaving said conjugate to release said biomolecule.

18. The method according to claim 17, wherein said biomolecule is a synthetic oligonucleotide and said modifying step is done during solid phase synthesis.

19. A method, which comprises:
modifying a biomolecule with the compound of claim 1 to produce a conjugate, wherein said conjugate contains a bioorthogonal moiety covalently linked to the biomolecule through a photolabile linker; and
reacting said conjugate with a solid support or another molecule functionalized with a complementary bioorthgonal moiety.

20. The method according to claim 19, wherein said biomolecule is a synthetic oligonucleotide and said modifying step is done during solid phase synthesis.

21. A method for isolating a biomolecule, comprising:
labeling a biomolecule with the compound of claim 1 to produce a conjugate, wherein said conjugate contains a bioorthogonal moiety covalently linked to the biomolecule through a photolabile linker;
reacting said conjugate with a solid support or another molecule functionalized with a complementary bioorthgonal moiety; and
releasing the biomolecule by exposing the solid support or other molecule to radiation.


SEQUENCE LISTING (1 sequence)

SEQ ID NO: 1 - DNA, 24 bp, Artificial Sequence
  Synthetic nucleotide
ggccgctacctctcaccactcatg